(19) United States
(12) Patent Application Publication
(10) Pub. No.: US 2008/0286871 A1
(43) Pub. Date: Nov. 20, 2008

(54) Modular genomes for synthetic biology and metabolic engineering

(76) Inventor: Robert A. Holt, North Vancouver (CA)

Correspondence Address: BC Cancer Agency;Technology Development Office, 675 West 10th Avenue, Vancouver, BC, V5Z 1L3, CA

(21) Appl. No.: 11/986,843

(22) Filed: Nov. 27, 2007

Related U.S. Application Data
(63) Continuation-in-part of application No. 11/546,609, filed on Oct. 12, 2006.

(51) Int. Cl. C12N 15/74; C12N 15/00; C12N 1/20; C12N 1/16

(52) U.S. Cl. 435/471; 435/440; 435/252.1; 435/252.8; 435/255.1

(57) ABSTRACT

The invention provides methods and compositions for assembling a modular replacement genome in a host microorganism. After such assembly, the host organism's genome is inactivated or ablated to permit full control of host cellular functions by the replacement genome. A modular replacement genome comprises an assembly of nucleic acid fragments, or segments, derived from one or more natural organisms or from synthetic polynucleotides or from a combination of both. Such an assembly, or set, of segments making up a replacement genome comprises a substantially complete set of genes and regulatory elements for carrying out minimal life functions under predefined culture conditions. The invention provides modular genomes having modules that are amenable to facile replacement, deletion, and/or additions. Such modules may be synthetic polynucleotides and may be designed for controlling gene content, excluding of genes that encode inhibitors or otherwise undesirable competing enzymes that divert a host cell from desired metabolic/synthetic processes; modifying codon usage to maximize or minimize protein production; modifying regulatory elements, including promoters, enhancers, repressors, activator, or the like, to modulate gene expression; balancing enzymatic and transport activities to optimize fluxes of substrates, intermediates, and products in metabolic pathways, and like objectives.

This application is a continuation-in-part of co-pending U.S. application Ser. No. 11/546,609 filed 12 Oct. 2006, which is hereby incorporated by reference in its entirety.

### FIELD OF THE INVENTION

The invention relates generally to methods and compositions for genetically modifying microorganisms, and more particularly, to methods and compositions for selectively modifying genomic regions containing predefined sets of genes and/or genetic elements.

### BACKGROUND

Metagenomics and the development of techniques for the facile production and manipulation of large fragments of DNA have spurred interest in engineering microorganisms for a host of important industrial and medical applications, e.g. Lorenz et al, Nature Reviews Microbiology, 3: 510-516 (2005); Branda et al, Developmental Cell, 6: 7-28 (2004); Kodumal et al, Proc. Natl. Acad. Sci., 101: 15573-15578 (2004); Tian et al, Nature, 432: 1050-1054 (2004). Metagenomics is the application of genomics techniques to the study of communities of microbial organisms directly in their natural environments, bypassing the need for isolation and laboratory cultivation of individual species, e.g. Reisenfeld et al, Annu. Rev. Genet., 38: 525-552 (2004). Such studies have revealed a wealth of genes encoding novel biochemical pathways and biocatalysts that potentially could play important roles in industrial processes, such as the extraction of fuels from refractory petroleum deposits, the conversion of agricultural raw materials into bulk and specialty chemicals, the generation of fuels from renewable resources, the discovery and development of therapeutically useful products, and the like, e.g. Handelsman, Microbiol. Mol. Biol. Rev., 68: 669-685 (2004); Van Hamme et al, Microbiol. Mol. Biol. Rev., 67: 503-549 (2003); Aitken et al, Nature, 431: 291-294 (2004). In particular, it is expected that metagenomics will provide an important source of raw materials for metabolic engineering, that is, the improvement of cellular activities by manipulation of enzymatic, transport, and regulatory functions of cells with the use of recombinant DNA technology, Bailey, Science, 252: 1668-1674 (1991).

In parallel with the above developments, there has been great interest in designing host organisms that are genetically well defined and, in some sense, minimal, not only for engineering applications, but also for understanding the basic life processes of free living microbes, e.g. Koonin, Annu. Rev. Genomics Hum. Genet., 1: 99-116 (2000); Kolisnychenko et al, Genome Research, 12: 640-647 (2002); Kobayashi et al, Proc. Natl. Acad. Sci., 100: 4678-4683 (2003); Glass et al, Proc. Natl. Acad. Sci., 103: 425-430 (2006); Posfai et al, Science, 312: 1044-1046 (2006); and the like. The primary approach in such studies has been to compare genomes of related species or strains to identify common genes and then to systematically delete genes or elements that are not shared, or to randomly disrupt genes and measure the effect on organism viability, e.g. Hutchinson et al, Science, 286: 2165 (1999). While reduced bacterial genomes appear to provide some advantages for protein production, the basic genome structure of such organisms remains unchanged from the essentially unordered assembly of genes, operons, and gene clusters provided by evolution, in which the genetic elements involved in every life function are scattered throughout the genome, Wolf et al, Genome Research, 11: 356-372 (2001); Bentley et al, Annu. Rev. Genet., 38: 771-792 (2004); Ochman et al, Curr. Opin. Microbiol., 6: 109-113 (2003); Ward et al, Curr. Opin. Microbiol., 8: 564-571 (2005). Such lack of order increases the difficulties of manipulating multiple genes and/or regulatory elements that may be involved in the same metabolic pathway.

In view of the above, it would be useful to have available a host organism for metabolic engineering that had a modular genome structure that permitted placement of functionally related genes and genetic elements in common regions and that allowed facile replacement of such regions, thereby taking advantage of recent advances in large-fragment polynucleotide synthesis and manipulation.

### SUMMARY OF THE INVENTION

The invention provides methods and compositions for assembling large nucleic acid constructs, such as modular replacement genomes in host microorganisms. After such assembly, the host organism's genome is inactivated or otherwise removed to permit full control of host cellular functions by the replacement genome. A modular replacement genome comprises an assembly of nucleic acid fragments, or segments, derived from one or more natural organisms or from synthetic polynucleotides or from a combination of both. Such an assembly, or set, of segments making up a replacement genome comprises a substantially complete set of genes and regulatory elements for carrying out minimal life functions under predefined culture conditions. “Substantially complete” in reference to the set of such genes and/or regulatory elements means that a minority of one or more genes and/or regulatory elements may be provided by episomal elements, such as plasmids, or the like, separate from the replacement genome. In one aspect, genes and regulatory elements of a replacement genome are selected to be substantially compatible with host genome counterparts. Such compatibility is usually accomplished by selecting a host genome-replacement genome pair whose sequences are sufficiently orthologous that (i) a substantial majority of replacement genes encode gene products or give rise to metabolites that do not interfere with host organism growth or replication, or are not otherwise poisonous to the host organism, and (ii) any subset of replacement genes giving rise to interfering or poisonous gene products or metabolites can be placed under inducible control.

In one aspect, component fragments, or segments, of a replacement genome are prepared separately in large-insert vectors wherein each different fragment is associated with one or more unique recombination elements. A plurality of such segment-containing vectors that collectively provide a complete sequence of a replacement genome is assembled stepwise in a predetermined order by repeated cycles of transformation of a host organism. In each successive cycle, a precursor replacement genome is formed that contains an additional segment, until a complete replacement genome is formed. Alternatively, a plurality of such segments may be assembled in parallel or in series by one or more cycles of co-transformation of a suitable host that permits self-assembly of multiple segments either by providing multiple segments with non-cross-reacting site-specific recombination elements or by the presence of a robust DNA repair system for joining double stranded breaks, e.g. as in D. radiodurans. After a complete replacement genome is formed, the host genome is then removed or rendered inoperable by inactivation, ablation or loss during cell partitioning, and any genes deleterious to the host are “turned on” by providing conditions for their induction, thereby forming a free-living synthetic cell with the replacement genome.

In another aspect, a replacement genome is modular in that after assembly and removal of the host genome, segments of the replacement genome may be selectively replaced with modified segments or added to by insertion of new segments using the recombinant elements left over from its initial assembly.

In another aspect, the invention provides a method of assembling a replacement genome in a host organism comprising the following steps: (a) providing a plurality of segments that cover a replacement genome, each segment being associated with one or more recombination elements; (b) transforming or co-transforming the host organism with one or more segments to form a precursor genome, the precursor genome being a recombinant of the one or more segments or a recombinant of a prior precursor genome and the one or more segments, such recombinant being formed by recombination of the recombination elements associated with the one or more segments; (c) repeating step (b) with segments of a predetermined ordering until the replacement genome is formed; and (d) removing the host genome. In one embodiment, the step of transforming or co-transforming may comprise co-transforming a host organism with the plurality of segments so that the replacement genome is formed in one step. In another embodiment, each end of each of the segments of a plurality has a unique overlapping sequence region with an end of another segment of the plurality so that the predetermined ordering is established upon formation of the replacement genome.

In another aspect, the invention provides a method of assembling a replacement genome in a host organism comprising the steps: (a) providing a plurality of segments that cover a replacement genome, each segment being associated with one or more recombination elements; (b) transforming the host organism with a segment to form a precursor genome, the precursor genome being the segment or a recombinant of the segment and a prior precursor genome, such recombinant being formed by at least one recombinase acting on the recombination elements associated with the segment; (c) repeating step (b) with each of a predetermined ordering of segments until the replacement genome is formed; and (d) removing the host genome. In one embodiment, each of the segments is associated with at least one recombination element selected from a plurality of different kinds of recombination elements and each successive segment in the predetermined ordering is associated with a different kind of recombination element selected from the plurality. In particular, the successive recombination elements may be mutant loxP sites that do not cross react. In one approach of such assembling process the step of transforming may produce a precursor genome containing a pair of recombination elements of the same kind, in which case, in one embodiment, the step of transforming may further include modifying one of said pair of recombination elements of the same kind so that it is reactive with a different kind of recombination element, such as the recombination element of the next segment. In another embodiment, each successive segment has at least one recombination element that comprises a first region of sequence homologous with a second region of sequence within the prior precursor genome, wherein such first and second sequences are from, or homologous to a sequence in, the replacement genome. In a particular embodiment, all of such sequences are identical. In still another embodiment, each successive segment has (i) a recombinant element comprising a first vector region that is homologous to a second vector region within the prior precursor genome, and (ii) a recombinant element that comprises a first region of sequence homologous with a second region of sequence within the prior precursor genome, wherein such first and second sequences are from, or homologous to a sequence in, the replacement genome. In still further embodiments, precursor genomes may be constructed in parallel in separate host organisms and then combined by transforming one of the hosts with one or more precursor genomes of one or more other hosts, where the precursor genomes are fused pair-wise into a single larger precursor genome, or a whole replacement genome, using conventional transformation techniques, such as polyethylene glycol-, or calcium ion-treated host organisms, or through protoplast fusion, or the like, e.g. Mercenier et al, Biochimie, 70: 503-517 (1988); Levi-Meyrueis et al, Mol. Gen. Genet., 179: 589-594 (1980); or like references. A replacement genome after assembly in one host may also be used to transform or replace the genome of separate host organisms using similar techniques.

In another aspect, the invention provides nucleic acid constructs made by serial site-specific recombination or serial homologous recombination, as well as host organisms in which they are assembled and replicated.

In still another aspect, the invention provides kits for assembling nucleic acid constructs from component polynucleotides, or segments, in a host organism, such kits comprising an ordered plurality, or minimal tiling set, of large-insert vectors, each large-insert vector being associated with one or more recombination elements and capable of incorporating an insert and each large-insert vector containing at least a first recombination element in common with its immediately preceding large-insert vector in the ordered plurality and at least a second recombinant element in common with its immediately succeeding large-insert vector in the ordered plurality, wherein such first and second recombination elements are different. In one aspect, each of such first and second recombination elements are site-specific recombination elements. In another aspect, each of such first and second recombination elements each have sequences homologous to a region of a component polynucleotide. In still another aspect, each large-insert vector of an ordered plurality has (i) a first vector recombination element homologous to a second vector recombination element in its immediately preceding large-insert vector, and (ii) a first recombination element homologous to a second recombination element in its immediately preceding large-insert vector, each first and second recombination elements being homologous to, or comprising, a portion of sequence of a component polynucleotide. In one embodiment of this aspect of the invention, the first and second vector recombination elements of each large-insert vector of an ordered plurality are each homologous to one another; and in another embodiment, each such vector recombination element has the same sequence. In another embodiment, each large-insert vector comprises a vector region and a component polynucleotide region, and each such vector region contains a selectable marker. Preferably, the selectable marker of each successive large-insert vector is different from that of the immediately preceding large-insert vector. More preferably, the selective marker of each large-insert vector is selected from a set of two different selectable markers and selectable markers in successive large-insert vectors alternate between such two selectable markers.

In another embodiment, kits of the invention include an ordered plurality of large-insert vectors, each large-insert vector being associated with one or more recombination elements and capable of incorporating an insert and each successive large-insert vector in the ordered plurality having at least one recombination element selected from a plurality of different kinds of recombination elements and wherein each successive large-insert vector in the ordered plurality is associated with a different kind of recombination element selected from the plurality. Such kits may further include reagents for carrying out recombination reactions of the assembly process, including at least one recombinase, buffers, co-factors, and the like.

The present invention advantageously addresses short comings of present technology by providing modular genomes having modules in the size range of several thousand basepairs, e.g. 5-10 kb, to many tens of thousands of basepairs, e.g. 50-100 kb, which are amenable to facile replacement, deletion, and/or additions. Such modules may be synthetic polynucleotides and may be designed for 1) controlling gene content, e.g. all of the genes in a multi-step metabolic pathway or numerous interacting or branching pathways may be contained on a single module, 2) excluding of genes that encode inhibitors or otherwise undesirable competing enzymes that divert a host cell from desired metabolic/synthetic processes, 3) modifying codon usage to maximize or minimize protein production, 4) modifying regulatory elements, including promoters, enhancers , repressors, activators, terminators, or the like, to modulate gene expression, 5) incorporating codons from non-natural amino acids, thereby making possible entirely new protein functions, 6) balancing enzymatic and transport activities to optimize fluxes of substrates, intermediates, and products in metabolic pathways, and like objectives.

### BRIEF DESCRIPTION OF THE DRAWINGS

FIGS. 1A-1G illustrates schematically several methods of assembling in a host organism donor genome segments into a replacement genome.

FIG. 2 illustrates alternating, or iterative, positive selection for successively using two positive selection markers in a segment assembly process.

FIG. 3 is a genetic map of a vector incorporating loxP recombination elements for incorporating donor genome segments into a growing precursor replacement genome.

FIG. 4 illustrates a recombination reaction between two single mutation loxP sites that results in a mutation-free site and a double mutation site in the recombinant product.

FIGS. 5A and 5B show genetic maps of large-insert vectors (fosmid cloning vectors) containing different selectable markers for carrying out methods of the invention. Each vector contains a unique blunt-end cloning site (SwaI) and unique restriction site (FseI) for linearization of clones prior to recombination.

FIG. 6 illustrates diagrammatically an embodiment of the invention for assembling large DNA circles in E. coli with the use of homologous recombination elements and a lambda Red recombination system. The ends of an incoming linear clone (ampicilin resistant) recombine with homologous sequence in a resident circular clone (kanamycin resistant). One region of end homology is between vectors (dotted) and the other between insert ends (light grey). Recombinants are selected according to the marker on the incoming clone (kanamycin).

FIGS. 7A and 7B show data confirming successful assembly of successive large-insert vectors in accordance with a method of the invention.

FIG. 8 is a two-dimensional electropherogram that shows pulsed field gel electrophoresis sizing of FseI linearized precursor and assembled clones described in Example III.

### DETAILED DESCRIPTION OF THE INVENTION

The practice of the present invention may employ, unless otherwise indicated, conventional techniques and descriptions of organic chemistry, molecular biology (including recombinant techniques), cell biology, biochemistry, and immunology, which are within the skill of the art. Such conventional techniques include, but are not limited to, vector construction, microbial host transformation, selection and application of genetic markers, manipulation of large polynucleotide fragments, preparation of synthetic polynucleotides, application of recombination systems, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies. A Laboratory Manual, Cells: A Laboratory Manual, PCR Primer: A Laboratory Manual, and Molecular Cloning. A Laboratory Manual (all from Cold Spring Harbor Laboratory Press), Stryer, L. (1995) Biochemistry (4th Ed.) Freeman, New York, Gait, “Oligonucleotide Synthesis. A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., Casali et al, editors, E. Coli Plasmid Vectors: Methods and Applications (Humana Press, Totowa, N.J., 2003), all of which are herein incorporated in their entirety by reference for all purposes.

In one aspect, the invention provides a method of constructing in a host cell a microbial genome by assembling in a predetermined order polynucleotide segments. The segments may be synthetic or may be derived from one or more donor organisms. In one aspect, such assembling is carried out by use of a recombination system that permits pairs of polynucleotides to be combined, or linked to one another, to form a single larger polynucleotide of a predetermined structure. Usually, members of such pairs are each double stranded replicatable polynucleotides, such as plasmids, phages, cosmids, BACs, or the like, or comprise portions of such replicatable polynucleotides. A recombination system comprises one or more recombinases, or other recombination enzymes, and one or more recombination elements, or nucleic acid sequences that are specifically recognized by a recombinase and/or are operated on or processed by recombination enzymes to provide joining sites between a pair of polynucleotides. Usually, reactions using a recombination system take place within a host cell. In some cases, a reaction using a recombination system may generate multiple products from which a desired product is selected, e.g. by selectable markers, size separation, or the like. In accordance with the invention, a sequence of recombination reactions is carried out such that in each successive reaction one member of a pair of polynucleotides to be combined is a product of the previous reaction. In certain embodiments, the resulting product is referred to as a “precursor replacement genome.” Thus, by such a sequence of reactions, polynucleotide segments making up a replacement genome are assembled segment by segment until a replacement genome is completed. A recombination system is selected so that each different segment is associated with one or more unique recombination elements thereby allowing each segment to be assembled in a predetermined order without interfering with previously assembled segments. Preferably, the same recombination system may be used after assembly to selectively replace, delete from, add to, or otherwise alter, segments of a completed replacement genome.

In one aspect of the invention, a plurality of segments are sequentially assembled to form a replicating nucleic acid construct inside of a host cell, wherein the size of the construct is larger than the expected size of conventionally handled genomic DNA, for example, that handled by conventional laboratory operations, such as, pipeting, mixing, stirring, transforming, and the like. In another aspect, the size of such construct is at least 500 kilobases (kb), or at least 600 kb, or at least 700 kb, or at least 800 kb. Typically, such constructs are not replacement genomes themselves, but may be precursor replacement genomes. In still another aspect, a plurality of segments collectively comprises a complete copy of a replacement genome; that is, the plurality of segments covers the replacement genome. Such coverage may or may not be redundant in that sequences of some segments of the plurality may overlap. In other aspects, such coverage is non-redundant in that there is no overlap among the sequences of segments in the plurality. A replacement genome may be derived directly from a genome of a natural strain or species of microorganism, which is referred to herein as a “donor genome.” A donor genome may be natural or unmodified, or it may have been modified to add, delete, substitute, or otherwise alter, genes, regulatory elements, operons, or other elements. Usually, segments in a plurality that comprise a donor genome are selected so that they collectively contain a substantially complete sequence of the donor genome and so that overlap is minimized; that is, each segment does not include any sequences that overlap with sequences of any other segment of the plurality, or if such overlap occurs, then it is minimal, for example, less than ten percent of either segment, or less than five percent of either segment, or less than two percent of either segment, or less than one percent of either segment. In other words, such segments, in some sense, represent a minimal tiling path of the donor genome. “Substantially complete” in reference to a set of segments derived from a donor genome means that the set contains sequences of the donor genome necessary for growth and replication under defined culture conditions. Thus, “substantially complete” includes situations where less than the entire genome sequence is included. In one aspect, “substantially complete” means at least 85 percent of a donor genome, or at least 90 percent of a donor genome, or at least 95 percent of a donor genome, or at least 98 percent of a donor genome. When a plurality of segments making up a donor genome is assembled in accordance with the invention it is referred to herein as a replacement genome. A replacement genome derived from a donor genome may be tailored later by selectively replacing segments containing the natural sequence components with synthetic polynucleotides in order to rearrange the ordering and/or composition of genes, regulatory elements, operons, gene clusters, and the like, for the purpose of enhancing performance of, or adding new functionality to, the resulting synthetic organism.

In some cases, during initial cycles of an assembly process, replication of a precursor replacement genome in a host organism may occur by operation of an origin of replication from a large-insert vector used in its construction, whereas replication in later cycles is controlled by an origin of replication derived from a donor genome. In other cases, immediately after or within the first few cycles of assembly, e.g. 1-2, or 2-4, or 3-6 assembly cycles, replication of a precursor replacement genome occurs by operation of an origin of replication derived from a donor genome. In such embodiments, early assembled segments, e.g. segments 1 to 4, or so, contain sequences comprising the donor origin of replication. In either case, a host is preferably selected so that the nucleic acids and proteins responsible for replication are substantially cross-functional with those encoded by the donor genome. That is, host organism proteins responsible for replication are operable with the donor origin of replication. In some embodiments, donor transcription factors may be added, e.g. via a host plasmid, to ensure that necessary proteins for precursor replacement genome replication are available. In some cases, other donor transcription factors may be provided to ensure availability of other necessary proteins, e.g. a particular donor replication protein for which there is no cross-functional host protein, or the like. Accordingly, in one aspect, preferably, a segment early in the assembly process (e.g. cycle 1-2, or 1-4, or 1-6) contains the replacement genome origin of replication (which may be a donor genome origin of replication) and all genes encoding necessary replication factors or proteins.

Likewise, whenever a donor genome contains genes that encode products that are incompatible with host organism growth and replication, then such genes, if indispensable, may be moved to the final segment in the assembly process; otherwise, if dispensable, such genes may be deleted. Optionally, such genes may be placed under inducible control, so that they may be activated after assembly is complete and the host genome and associated support functions are no longer required. In one aspect, donor genes (or other sequences) incompatible with a host organism can be identified by whole genome shotgun sequencing using conventional techniques, e.g. Weber et al, Genome Research, 7: 401-409 (1997); Adams et al, Science, 287: 2185-2195 (2000); Waterston et al, Proc. Natl. Acad. Sci., 99: 3712-3716 (2002); Reed et al, J. Virol. Meth., 129: 91-96 (2005); and the like, which references are incorporated by reference. Briefly, in whole genome shotgun sequencing, genomic DNA of a donor organism is randomly sheared, cloned in small-, medium-, and large-insert expression vectors, transformed into the host organism, and randomly selected clones are sequenced. A sufficient amount of sequencing is carried out so that if gaps remain in the assembled donor genome, it must be attributed to the presence of a cloned sequence that is incompatible with the host organism, and not with under sampling of donor genome fragments. The incompatible donor sequences are identified by the locations of the gaps in the assembled donor genome. Alternatively, or as a confirmation, individually selected donor sequences may be tested by introducing into a host in a suitable expression vector.

In one aspect, a host organism and donor organism are selected so that proteins responsible for replication are substantially cross-functional, thereby allowing replication of precursor replacement genomes by host replication proteins. One approach to accomplish this is selection of host organisms and donor organisms that are close evolutionarily, e.g. as measured by genome sequence homology, particularly of core function genes, e.g. those involved with replication, transcription, protein synthesis, substrate transport, energetic metabolism, cell division, and the like. Guidance for selecting core function genes for determining a measure of cross-functionality of encoded proteins may be found in an extensive literature on minimal microbial genomes, e.g. as represented by the following references that are incorporated by reference: Koonin, Annu. Rev. Genomics Hum. Genet., 1: 99-116 (2000); Kobayashi et al, Proc. Natl. Acad. Sci., 100: 4678-4683 (2003); Glass et al, Proc. Natl. Acad. Sci., 103: 425-430 (2006); Gil et al, Microbiol. Molecular Biol. Rev., 68: 518-537 (2004); U.S. Pat. No. 6,673,567; U.S. Pat. No. 6,207,384; and the like. In one aspect, core function genes of host and donor genomes are at least 50 percent homologous; in another aspect, core function genes of host and donor genomes are at least 60 percent homologous; or at least 70 percent homologous, or at least 80 percent homologous, or at least 90 percent homologous, or at least 95 percent homologous, or at least 98 percent homologous. In one aspect, core function genes for determining cross-functionality are genes encoding proteins necessary for genome replication. Whenever Escherichia coli (E. coli) is employed as a host organism, core function genes for genome replication are well known to those of ordinary skill in the art, as evidence by the treatise: Kornberg and Baker, DNA Replication, Second Edition (Freeman, San Francisco, 1992), which is incorporated by reference. In one aspect, such genes include those encoding polymerases, primases, ligases, helicases, and gyrases. In another aspect, such genes are selected from the following set: dnaA, dnaB, dnaC, dnaE, dnaG, dnaJ, dnaK, dnaN, dnaQ, dnaT, dnaX, dnaY, dnaZ, dut, grpE, gyrA, gyrB, lig, nrdA, nrdB, ori, polA, polB, priA, priB, priC, rep, mhA, rpoA, rpoB, rpoC, rpoD, ssb, ter, topA, trxA, and tus, where these gene designations are defined in Kornberg and Baker (cited above), and are defined by sequence in publicly available databases, such as NCBI, Ensembl, GenBank, or the like. In particular, the sequence of E. coli strain K-12 is disclosed in Blattner et al, Science, 277: 1453-1474 (1977), which is incorporated by reference. Whenever Bacillus subtilis is employed as a host organism core function genes for genome replication may be selected from the following set: dnaA, dnaB, dnaC, dnaD, dnaE, dnaF, dnaG, dnaH, dnaI, dnax, gyrA, gyrB, nrdA, and the like.

Alternatively, activities of selected host proteins in carrying out donor functions may be assessed empirically where assays are available, e.g. a donor origin of replication may be cloned into a host using a vector whose own origin of replication is under conditional control, e.g. temperature sensitive control, so that upon disablement of the vector origin, cross-functionality may be tested by assessing whether and to what extent the vector replicates (by use of host replication proteins).

Replacement genomes may be assembled in accordance with the invention in a wide variety of host organisms. Preferably, the host organism is a prokaryotic organism. In one aspect, the host organism is a bacterium, and more usually, an enteric bacterium, such an E. coli. In another aspect, the host organism is a Bacillus subtilis. Preferably, the donor organism is a prokaryotic organism. In one aspect, the donor organism is a bacterium. Exemplary donor organisms for use with an E. coli host include Hemophilus, and more particularly, Hemophilus influenzae, Pseudomonas, and more particularly, Pseudomonas putida.

In one aspect, segments used in assembling a replacement genome are cloned or constructed using conventional techniques in conventional cloning vectors, including plasmids, phages, cosmids, and/or bacterial artificial chromosomes (BACs) and P1-derived artificial chromosomes (PACs), P1 vectors, and the like. In order to minimize assembly steps, preferably, most, if not all, segments are provided as inserts of large-insert cloning vectors, such as BACs or PACs. After assembly of a replacement genome is completed, the type of vector used and the sizes of replacement fragments for further alterations will depend on particular applications. A large-insert vector is a vector capable containing an insert having a length in the range of from 50 kb to 300 kb, or greater, and transforming a prokaryotic host organism, such as a bacteria. In particular, a large number of BACs are available for use in RecA⁻ E. coli host organisms. In one aspect, a set of segments for assembling a replacement genome may comprise inserts in the same type of cloning vector or in different types of cloning vectors, e.g. a majority may comprise large fragments, i.e. greater than 100 kb, in BACs and a minority may comprise smaller fragments in other cloning vectors, such as phages, comids and/or plasmids. In another aspect, BACs are employed as the primary cloning vector for segments of a replacement genome; that is, a majority of the sequence of a replacement genome is provided in BACs. Lengths of segments in a plurality may vary widely depending on several factors including the size of a donor genome, the desirability of minimizing steps in the assembly process, the desired arrangement of genes, operons, gene clusters and the like, anticipated segment substitutions (for example, it may be more efficient to use several smaller synthetic segments separately rather than a single large synthetic segment), and so on. In one aspect, segments have lengths in the range of from a few thousands of basepairs, e.g. 2-10 kb, to several hundreds of thousands of basepairs, e.g. 100-300 kb. In another aspect, segments have lengths in the range of from tens of thousands of basepairs, e.g. 10-50 kb, to hundreds of thousand basepairs, e.g. 100-300 kb. Preferably, segments are cloned in BAC vectors, which are described in the following references that are incorporated by reference: Zhao et al, editors, Bacterial Artificial Chromosomes (Humana Press, Totowa, N.J., 2004); Kim et al, Genomics, 34: 213-218 (1996); Shizuya et al, Proc. Natl. Acad. Sci., 89: 8794-8797 (1992); U.S. Pat. Nos. 5,874,259 and 6,472,177; and the like. Techniques for assembling inserts into BACs from several smaller pieces are well known in the art, as evidenced by the following reference: O'Connor et al, Science, 1307-1312 (1989), which is incorporated by reference. Exemplary vectors that may be used with the invention, with no or minor modifications, include pBeloBAC11, pBACe3.6, pCClBAC, pSMART VC, pIndigoBAC-5, SuperCos 1, and the like, which are commercially available or described in GenBank.

Assembly of a replacement genome may be carried out using a variety of techniques, including the use of restriction endonucleases and ligases for inserting fragments, the use of hosts having robust DNA repair mechanisms, and the use of recombination based methods for site-specific insertion. Site-specific recombination systems for use with the invention include at least one recombinase that usually operates on a pair of reactive recombination elements (or sites) to catalyze strand scission and rejoining. Reactive recombination elements are usually, but not necessarily, recombination sites having identical sequences. Recombination systems may also include additional ancillary proteins that may be operationally associated with a recombinase. In one aspect, in order to sequentially assemble different segments into a growing precursor replacement genome, a recombinase is selected that is capable of catalyzing separate recombination events with recombination elements having different sequences without the occurrence of significant cross reaction among different recombination elements. Thus, in one approach, a sufficient number of different non-cross reacting recombination elements must be available for complete assembly of a replacement genome. In another approach, non-cross reacting recombination elements may be re-used in alternating steps of assembly; thus, only two non-cross reacting recombination elements are required. Many recombination systems are useful in the present invention and may be used alone or in combination with one another. Suitable recombination systems include, but are not limited to: 1) linear homologous recombination using two crossover sites near the ends of the sequence of interest, exemplified by a Red/ET, or lambda Red, system (Gene Bridges GmbH, Dresden, Germany); 2) circle homologous integration followed by a second resolving recombination, exemplified by Cre-lox or flp-frt sites in a recombination mediated cassette exchange (RMCE) approach; 3) linear, sequence-specific recombination (e.g., via a phage integrase such as λ or phiC31); and 4) sequence-specific circle integration. Exemplary site-specific and homologous recombination systems include, but are not limited to, Cre-loxP, Flp-FRT, att-Int (Gateway), Red/ET (lambda Red), RecA, and the like. These and other recombination systems are well-known to those of ordinary skill in the art and are described in the following references, which are incorporated by reference: Branda et al, Developmental Cell, 6: 7-28 (2004); Baer et al, Curr. Opin. Biotech., 12: 473-480 (2001); Sauer, Nucleic Acids Research, 24: 4608-4613 (1996); Yu et al, Proc. Natl. Acad. Sci., 97: 5978-5983 (2000); Lee et al, Genomics, 73: 56-65 (2001); Muyrers et al, EMBO Rep., 1: 239-243 (2000); Cheo et al, Genome Research, 14: 2111-2120 (2004); Missirlis et al, BMC Genomics, 7: 73 (2006); U.S. Pat. Nos. 6,509,156; 6,355,412; 6,465,254; 6,720,140; 5,776,449; 5,888,732; and the like. Recombinases may be provided by expression of genes that may be carried by the host genome, or by an episome, such as a plasmid, or by one or more segments of a precursor replacement genome. Preferably, expressions of recombinases are under inducible control in order to minimize the occurrence of spurious or undesired recombination during the assembly process. Also, preferably, a host organism is selected that is free of recombination elements used in the replacement genome (or DNA circle) assembly process, or a selected organism is treated to remove or disable such elements to prevent spurious or unintended recombination reactions.

In one aspect, the assembly process of the invention includes successive steps of recombining in a host organism a new segment of a replacement genome with segments that have previously been assembled, and which constitute a precursor replacement genome. Such steps are carried out using conventional vectors and transformation techniques in conjunction with a recombination system, such as one of those indicated above. Typically, each such step includes substeps of transforming the host with a vector containing a new segment operationally associated with one or more unique recombination elements, culturing transformed host organisms, and selecting host organisms containing recombinants, i.e., precursor replacement genomes that have successfully recombined with a new segment to generate a successive precursor replacement genome (or a completed replacement genome), as the case may be. In some embodiments, multiple segments may be recombined with a precursor replacement genome in a single cycle, e.g. using the approach of Church et al, International patent publication WO 2006/055836, which is incorporated herein by reference.

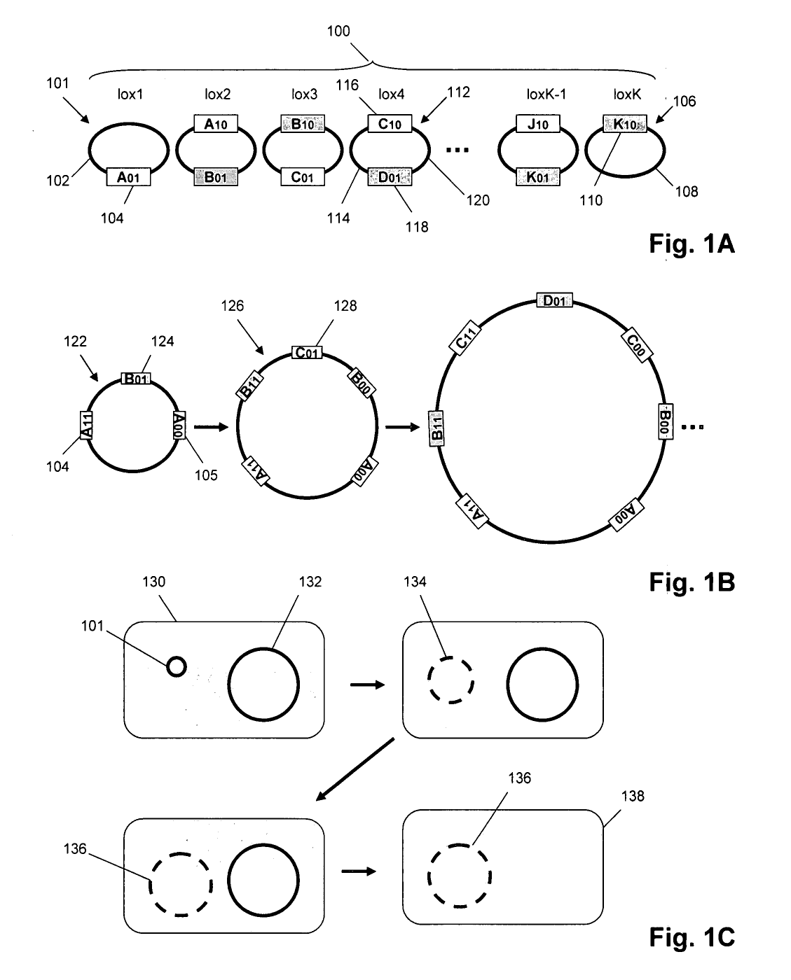

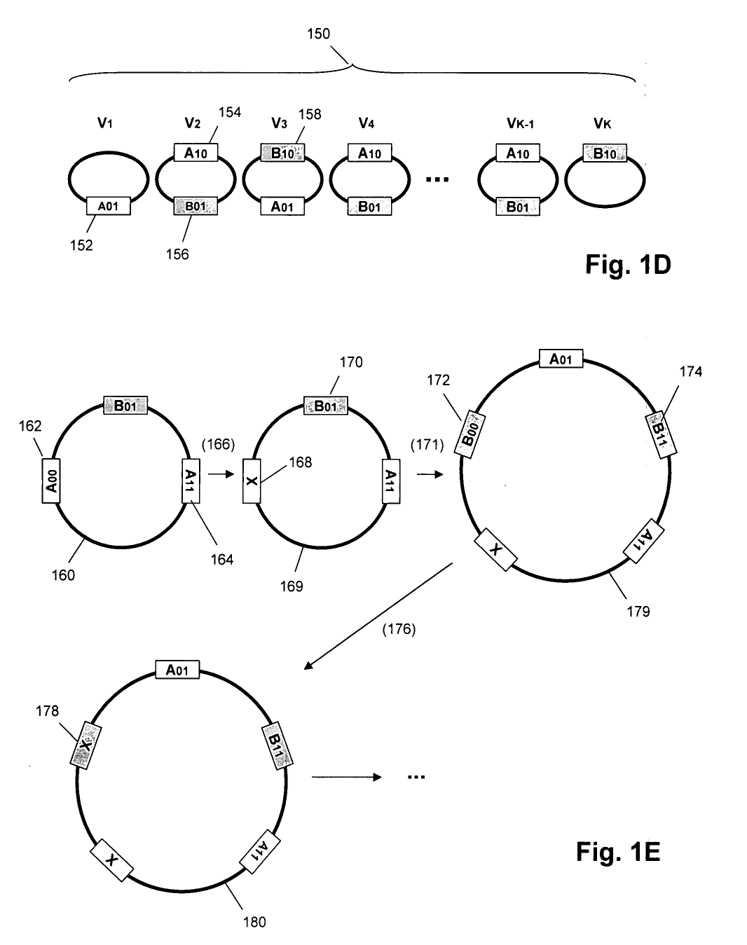

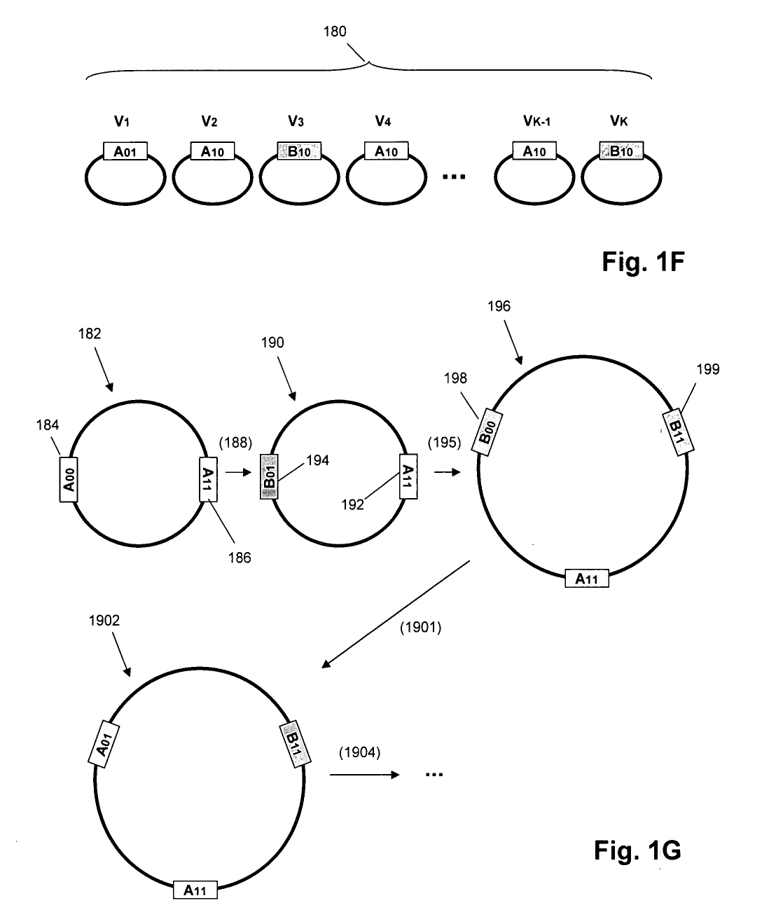

In one aspect of the invention, segment assembly is carried out with site-specific recombination, as illustrated in FIGS. 1A-1G. Site-specific recombination elements are selected and arranged in vectors to drive recombination reaction to the desired products. Sets of site-specific recombination elements are provided that (i) have substantially no cross reactivity with one another, and (ii) are oriented so that stable integration occurs in each step of the assembly process. As reviewed by Branda et al (cited above), for example, the λ integrase family of site-specific recombination elements, which include loxP and FRT, share a common mechanism of DNA recombination that involves strand cleavage, strand exchange, and ligation. Although distinct at the nucleotide level, loxP and FRT sites share an overall structure which includes two 13 basepair palindromic sequences, or inverted repeats, separated by an 8 basepair asymmetric core, or spacer, sequence. In the presence of two sites, recombinase monomers bound to the inverted repeats promote the formation of a synaptic complex and recombination between the two sites. Strand cleavage, exchange, and ligation occur within the spacers. Because of spacer asymmetry, strand exchange is possible only when target sites are connected by synapses in one orientation. Consequently, the relative orientation of target sites with respect to one another determines the outcome of recombination: Cre and Flp recombinases will excise a circular molecule from between two directly repeated target sites, integrate a circular molecule into a linear molecule each possessing a target site, invert the DNA between two inverted sites, and exchange sequences distal to target sites present on two linear molecules. Because insertion ordinarily leaves two identical sites in cis configuration, which are themselves substrates for recombination, stable insertions are difficult or impossible using two wild type sites. However, many recombinases, such as Cre and Flp, tolerate certain variations in their target sequences and effectively catalyze recombination only between certain subsets of the alternative sites. This property is exploited to permit successive recombination events for replacement genome assembly. Variant target sites for λ intergrase recombinases, such as Cre and Flp, fall into two classes: spacer variant and invert-repeat variants. The first class contains nucleotide substitutions within the spacer sequence and exploits the finding that it is spacer length, not sequence that is the critical factor for efficient recombination, so long as the sequence between participating sites is identical. Recombination is therefore efficiently mediated between pairs of homotypic (e.g. FRT/FRT or F₃/F₃) but not heterotypic (e.g. FRT/F₃) sites. The second class of alternative sites (inverted repeat variants) may also be exploited to provide stable insertions. A target site containing a nucleotide substitution in the “left side” inverted repeat (an “LE” mutant site) can recombine with a site containing an analogous substitution in the “right end” inverted repeat (an “RE” mutant site), although at a slower reaction rate than wild type sequences. Such mutants are designed so that the recombination product harbors one wild type site and one LE/RE double mutant site, the latter being effectively inert. Thus, insertion with such single mutant LE and RE sites results in the formation of only one potentially active recombination element, which itself may be inactivated or modified and used for subsequent insertions. These concepts are illustrated in FIGS. 1A-1G for several embodiments of the invention.

FIG. 1A illustrates a plurality of segments (100) carried in vectors, lox1 through loxK, that each have a unique combination of site-specific recombination elements labeled “A₀₁,” “A₁₀,” “B₀₁,” “B₁₀,” “C₀₁,” . . . “K₁₀,” where each different letter, “A,” “B,” etc., indicates a different non-cross-reacting site-specific recombination element, and where subscripts “01” and “10” indicate a recombination element has an RE mutant site (“01”) or an LE mutant site (“10”). Correspondingly, a letter with subscripts “11” indicates a double mutant site and a letter with subscripts “00” indicates a mutant-free site. It is noted that the diagrams of vectors are only symbolic representations and are not to scale or proportion. For example, even though the site-specific recombination elements are shown at opposite sides of the vectors, this is not a required configuration. The recombination elements may be juxtaposed or they may be interspersed in the vector or segment. Vector lox1 (101) containing initial segment (102) requires only a single recombination element “A₀₁” (104) in this embodiment. Likewise, vector loxK (106) containing the final segment (108) requires only a single recombination element “K₁₀” (110). Vectors lox2 through loxK-1 each have two different recombination elements, as exemplified by vector lox4 (112), which comprises segment (114), recombination element “C₁₀” (116), recombination element “D₀₁” (118), and portion (120), which may be part of segment (114) or simply a connection between the two recombination elements (e.g. a bond connecting two adjacent sequences or an intervening polynucleotide). In this embodiment, each of the segments-containing vectors of plurality (100), except for the first (lox1) and the last (loxK), contains at least a first recombination element (e.g. “A₁₀” of lox2) in common with its immediately preceding vector (i.e. “A₀₁” of lox1) in the predetermined order shown and at least a second recombinant element (e.g. “B₀₁” of lox2) in common with its immediately succeeding vector (i.e. “B₁₀” of lox3), wherein such first and second recombination elements are different (i.e. in this embodiment, the first is type “A” and the second is type “B”). Again, an important property of the different types (or kinds) of recombination elements is that members or variants of one type (or kind) do not cross react (or substantially do not cross react) with members or variants of another type (or kind). As illustrated in FIG. 1B, segments of plurality (100) are assembled stepwise by adding them on segment at a time to form a succession of precursor replacement genomes. In one aspect, each step in the assembly process comprises a cycle of steps (or substeps) including transforming a host and selecting a resulting transformant using a selectable marker. The relative ordering of recombination elements is shown in first recombinant (122), which is the first precursor replacement genome. Since site-specific recombination is conservative, in that DNA synthesis is not required and sequences are neither lost nor gained in the reaction, first recombinant (122) contains pieces of all the recombination elements of the two vectors that were combined, i.e. two copies of an “A” type recombination element in double mutant form (104) and in mutant-free form (105), and one copy of recombination element “B₀₁” (124), which serves as the unique recombination site for the next vector, lox3. After transformation, recombination and selection, the resulting host harboring first recombinant (122) is transformed with vector lox3 to form the next recombinant, or precursor replacement genome (126). Again, sequences are conserved in the recombinant of (122) and lox3 so that precursor replacement genome (126) contains five recombination elements: “A₀₀” (active), “A₁₁” (inert), “B₀₀” (active), “B₁₁” (inert), and “C₀₁” (128), which again is the unique recombination site for the next vector, lox4. The process continues until a replacement genome is complete. The ordering of the active recombination elements (“A₀₀,” “B₀₀,” “C₀₀,” etc.) relative to the inert recombination elements (“A₁₁,” “B₁₁,” “C₁₁,” etc.) may be varied by changing the ordering of the LE and RE mutant sites in vectors (100). For example, if lox2 contained “B₁₀” and lox3 contained “B₀₁,” then the positions of “B₁₁” and “B₀₀” would be swapped and the resulting vector corresponding to (126) would have inert site “A₁₁” sandwiched between “A₀₀” and “B₀₀.”. As indicated, in this embodiment, recombination takes place in a host cell, such as illustrated diagrammatically in FIG. 1C. Host organism (130) is transformed by initial vector (101) to form a host containing a host genome (132) and vector (101). In subsequent cycles of the assembly process, successively larger recombinants (134), i.e. precursor replacement genomes, are formed until a completed replacement genome (136) is present. Host genome (132) is then removed or ablated to give synthetic cell (138) containing only replacement genome (136).

As discussed more fully below, the above process may be carried out with pairs of LE and RE mutant recombination elements for each type, “A” through “K,” as taught by Missirlis et al, BMC Genomics, 7: 73 (4 Apr. 2006), which is incorporated by reference. Briefly, LE and RE mutant pairs are prepared for each type of recombination element. When a recombination event occurs (e.g., part of element “B10” on lox3 is combined with element “B01” on lox3), both mutants are present in only one of the product sites, and the other product site is free of mutations. This results directly in a modular replacement genome. That is, the operable recombination sites may be used with the recombination system employed to exchange segments for modifying the properties of the synthetic organism, e.g. using a RMCE procedure.

In another aspect, segments may be assembled into a replacement genome by using fewer recombinations elements, as illustrated in FIGS. 1D and 1E. A plurality of segments is provided in vectors V₁ through V_{K} (150). In this embodiment, the vectors do not each have one or more unique recombination elements; instead, the recombination elements are re-used in alternating cycles of segment incorporation. Such re-cycling of recombination elements may be accomplished with a plurality of different types of recombination elements, each of which is provided as a pair of single mutants that may recombine with each other to produce an active mutant-free form and an inert double mutant form. Such a plurality of different recombination elements may contain two, three, four, five, six, seven, eight recombination elements. In one aspect, recombination elements may be conveniently introduced into BACs carrying the segments by Red/ET recombination, e.g. as disclosed in U.S. Pat. No. 6,509,156; and Yu et al, Proc. Natl. Acad. Sci., 97: 5978-5983 (2000); and/or using reagents commercially available from GeneBridges GmbH (Dresden, Germany). In particular, using appropriate host bacteria, sequences to be inserted into a BAC may be prepared by PCR, where the resulting amplicon contains unique flanking sequences of 30-50 basepairs. Such amplicons are recombined with regions of the BAC bounded by the same unique sequences.

In FIG. 1D, vector V₁ containing the first segment and vector V_{K} containing the last segment, V_{K}, each have a single recombination element, and the rest of the vectors, V₂ through V_{K-1}, each have two. Recombination element A₀₁ (152) on V₁ is a single mutant site that is operable with recombination element A₁₀ (154) on V₂, which is a different single mutant site. Likewise, recombination element B₀₁ (156) on V₂ is a single mutant site that is operable with recombination element B₁₀ (158) on V₃, which is a different single mutant site. The same four sites may be used with all of the vectors V₁ through V_{K}, when used as follows. V₁ and V₂ are transformed into a Red/ET competent host that also expresses an appropriate recombinase to form recombinant (160), in which recombination elements A₀₁ (V₁) and A₁₀ (V₂) are changed to functional A₀₀ site (162) and non-functional A₁₁ site (164). An amplicon is prepared containing 30-50 basepair flanking sequences that are identical to sequences flanking A₀₀ (162) on recombinant (160). The host bacteria containing recombinant (160) is transformed with the amplicon so that it can recombine (166) with the portion of recombinant (160) containing A₀₀ (162) to produce recombinant (169), which is shown to have a disabled recombination site “X” (168). An advantage of the Red/ET system is that recombinants can be detected by PCR; growth on a selective medium is not required. Modified recombinant (169) may then be used in the next assembly step by transforming its host with vector V₃ containing a third segment and recombination elements B₁₀ and A₀₁ to form (171) recombinant (179), which contains a functional B₀₀ site and a non-functional B₁₁ site. As above, after selection of a recombinant (179), the functional B₀₀ site is disrupted (176) to form modified recombinant (or precursor replacement genome)(180). Assembly of a replacement genome continues in a similar manner for the remaining segments.

A recombination system, such as Red/ET may also be used as illustrated in FIGS. 1F-1G to modify an undesired functional recombination element within a segment-addition cycle. A plurality of vectors (180) is provided that each contains only one single mutant form of one type of recombination element. As above, only two types recombination elements are shown in the embodiment of FIG. 1F (A's and B's); however, further types of recombination elements may be employed in alternative embodiments. Vectors V₁ and V₂ are recombined to form recombinant (182) that contains active recombination element A₀₀ (184) and inert recombination element A₁₁ (186). In this embodiment, instead of inactivating element A₀₀, a homologous recombination system, such as Red/ET, is employed to exchange the active recombination element A₀₀ with a recombination element complementary to the element of the next vector to be inserted. (As above, this allows the type A recombination elements to be re-used in subsequent steps). After such exchange (188), precursor replacement genome (190) is formed that has one inactive recombination element A₁₁ (192) and one active recombination element B₀₁ (194). Precursor replacement genome (190) is then combined with vector V₃ so that element B₀₁ recombines (195) with element B₁₀ of vector V₃ to form precursor replacement genome (196) containing active B₀₀ (198) and inert B₁₁ (199). In the next step, B₀₀ is exchanged (1901) with A₀₁ to produce precursor replacement genome (1902). Similar cycles (1904) of transforming to add a segment and transforming to exchange a recombination element are carried out until a replacement genome is assembled.

As mentioned above, assembly of nucleic acid constructs, DNA circles, or replacement genomes may also be carried out in part or wholly in parallel by partial or complete self-assembly after co-transformation. In one aspect of this embodiment, multiple segments are co-transformed into a host organism wherein each segment is associated with a site-specific recombination element that does not cross-react with its co-transforming segments, so that recombination results in correctly ordered segments in the nucleic acid construct produced, whether it is a precursor replacement genome or a completed replacement genome. Segments may contain multiple independent selective markers so that successful co-transformants can be identified. Suitable non-cross reacting recombination elements may be selected from the group of mutant loxP recombination elements disclosed below. In another aspect of this embodiment, a plurality of segments having unique overlapping ends of identical or homologous sequences (i.e. each overlapping region comprises unique sequences, or at least distinct from other overlapping regions) may be assembled in parallel by co-transforming into a suitable host having a robust DNA repair system, such as Deinococcus radiodurans, which has been studied for potential applications in bioremediation of radioactively contaminated environments, e.g. Zahradka et al, Nature, 443: 569-573 (2006); Makarova et al, Microbiol. Mol. Biol. Rev., 65: 44-79 (2001); Brim et al, Nature Biotechnology, 18: 85-90 (2000); Langer et al, Nature Biotechnology, 16: 929-933 (1998); Narumi et al, U.S. Pat. No. 6,770,476; which references are incorporated herein by reference. Suitable overlapping sequences may have lengths of from a few hundred basepairs, e.g. 100-1000, to several thousand basepairs, e.g. 1000-20,000. In still another embodiments, a plurality of segments having unique overlapping ends of identical or homologous sequences as above may be assembled in parallel by co-transforming into a suitable host having a homologous recombination system that recombines the overlapping ends, such as a Red/ET system, or the like.

Generally, and in the particular examples above, transforming host microorganisms with vectors carrying segments is carried out with conventional techniques. As used herein, the terms “transformation” and “transfection” are intended to refer to a variety of art-recognized techniques for introducing an exogenous nucleic acid sequence (e.g., DNA) into a host cell, including calcium phosphate or calcium chloride co-precipitation, DEAB-dextran-mediated transfection, lipofection, electroporation, optoporation, mechanical injection, biolistic injection, and the like. Suitable methods for transforming or transfecting host cells are found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and like laboratory manuals.

Transformed microorganisms, that is, those containing recombinant molecules, may be selected with a variety of positive and/or negative selection methods or markers. In certain aspects, the positive selection marker is a gene that allows growth in the absence of an essential nutrient, such as an amino acid. For example, in the absence of thymine and thyimidine, cells expressing the thyA gene survive, while cells not expressing this gene do not. A variety of suitable positive/negative selection pairs are available in the art. For example, various amino acid analogs known in the art could be used as a negative selection, while growth on minimal media (relative to the amino acid analog) could be used as a positive selection. Visually detectable markers are also suitable for use in the present invention, and may be positively and negatively selected and/or screened using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodaminie, dichiorotriaziniylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucoriniidases, phosphatases, peroxidases, cholinesterases and the like. In other aspects, the positive selection marker is a gene that confers resistance to a compound which would be lethal to the cell in the absence of the gene. For example, a cell expressing an antibiotic resistance gene would survive in the presence of an antibiotic, while a cell lacking the gene would not. For instance, the presence of a tetracycline resistance gene could be positively selected for in the presence of tetracycline, and negatively selected against in the presence of fusaric acid. Suitable antibiotic resistance genes include, but are not limited to, genes such as ampicillin-resistance gene, neomycin-resistance gene, blasticidin-resistance gene, hygromycin-resistance gene, puromycin-resistance gene, chloramphenicol-resistance gene and the like. In certain aspects, tie negative selection marker is a gene that is lethal to the target cell in the presence of a particular substrate. For example, the thyA gene is lethal in the presence of trimethoprim. Accordingly, cells that grow in the presence trimethoprim do not express the thyA gene. Negative selection markers include, but are not limited to, genes such as thyA, sacB, gnd, gapC, zwJ, talA, taiB, ppc, gdhA, pgi, Jbp, pykA, cit, acs, edd, icdA, groEL, secA and the like.

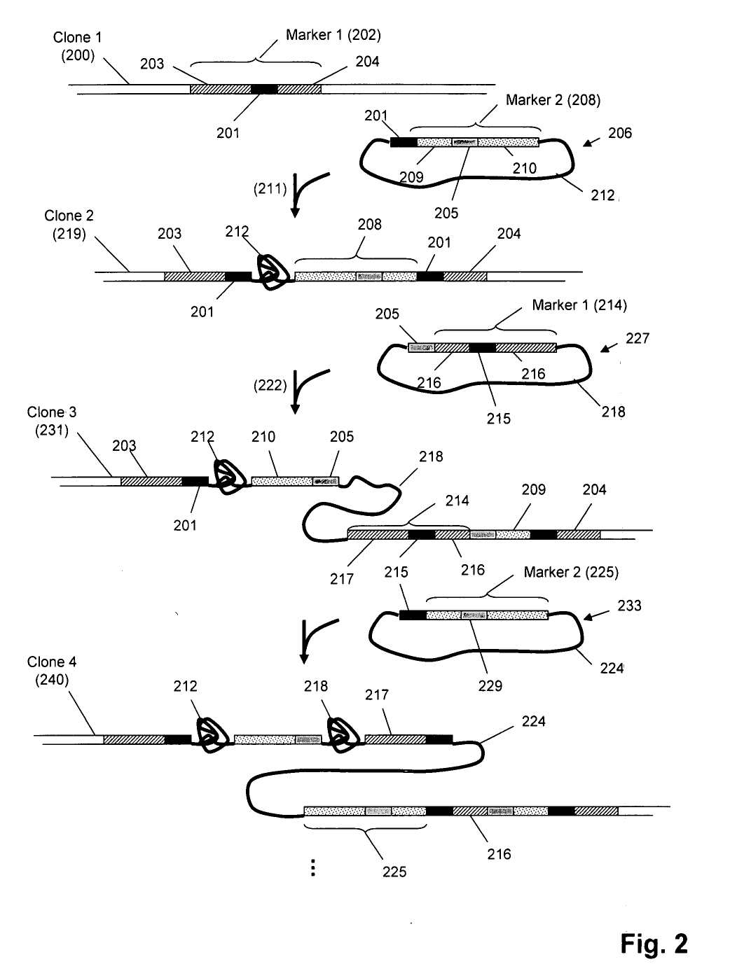

Selection methods and/or markers may be used efficiently in a multi-step assembly process, such as called for by the invention, by employing a pair of selection methods or markers that are switched, or used reciprocally, between successive recombination steps, e.g. as taught by O'Connor et al, Science, 244: 1307-1312 (1989); Kodumal et al, Proc. Natl. Acad. Sci., 101: 15573-15578 (2004); or the like. For example, as illustrated diagrammatically in FIG. 2 a first segment-containing vector (200) is transformed (211) into a host containing a recombination element (201) embedded in a gene for a positive selective marker (“Marker 1”) (202), e.g. SacBII. The SacBII gene codes for an enzyme that converts sucrose to levansucrase, which is toxic to bacterial cells (see Pierce et al., Proc. Natl. Acad. Sci 89; 2056-2060. 1992). Element (201) divides marker (202) into two parts (203) and (204). A successive segment-containing vector (206) is transformed (211) into the host containing a recombination element (201) complimentary to that in the prior recombinant, and a second non-complementary recombination element (205) embedded in a second selectable marker gene (“Marker 2”)(208) (for example, the tetracycline resistance gene, which confers sensitivity to fusaric acid, e.g. Bochner et al., J. Bacteriology 143; 926-933. 1980). As above, recombination element (205) divides Marker 2 gene (208) into two parts (209) and (210). Recombination between clone 1 (200) and vector (206) containing segment (212) forms clone 2 (219) that has a disrupted marker 1 gene (see the separation of parts (203) and (204)), but a fully functional tetracycline resistance gene (208) containing recombination element (205); thus, when plated on solid media containing sucrose, only recombinant clones will grow. Clone 2 (219) may then be recombined (222) with vector (227) containing (i) complementary recombination element (205), (ii) marker 1 gene (214) containing embedded recombination element (215) (which divides marker 1 into parts (216) and (217)), and segment (218). As above, recombination element (205) of vector (227) reacts with element (205) of clone 2 (219) to form clone 3 (231), in which marker 2 (208) of clone 2 is disrupted by the separation of parts (209) and (210), but which also contain a functional marker (214) that permits selection of recombinants by exposure to tetracycline. Functional marker (214) also contains embedded recombination element (215). Segments (212) and (218) are contained clone 3. Assembly continues by providing vector (233) that contains the next segment (224) and a marker 2 gene (225) containing another embedded recombination element (229) as well as a complementary recombination element (215) for insertion into clone 3. Vector (233) and clone 3 (231) form recombinant clone 4 (240) and three segments, the final one of which (224) disrupts marker 1 gene (214) (by splitting its coding region, see (216) and (217)) and provides new operational marker 2 gene (225). Alternating disruptable positive selection markers in this manner allows stepwise accrual of donor genome segments in the host organism. A disruptable positive selection marker may also comprise a recombination element that is positioned between a marker gene and its promoter site.

After a replacement genome is assembled and becomes operable in a host organism, the host genome is removed or ablated. In one aspect, it is removed by creating conditions that select against host organisms that retain the host genome. In another aspect, ablating the host genome includes creating conditions that both select for the replacement genome, e.g. via antibiotic resistance markers, and select against the host genome, e.g. inserting an inducible SacB, or like gene, in the host genome. In still another aspect, in certain selections of host and donor genomes, restriction endonucleases are available that cleave host genome DNA but not donor genome DNA. In such circumstances, providing an inducible gene that expresses such a restriction endonuclease may be provided to remove a host genome. For example, the restriction enzyme FseI cleaves at four sites in E. coli, but none in H. influenzae.

### Mutant LoxP Sites for Serial Site-Specific Recombination

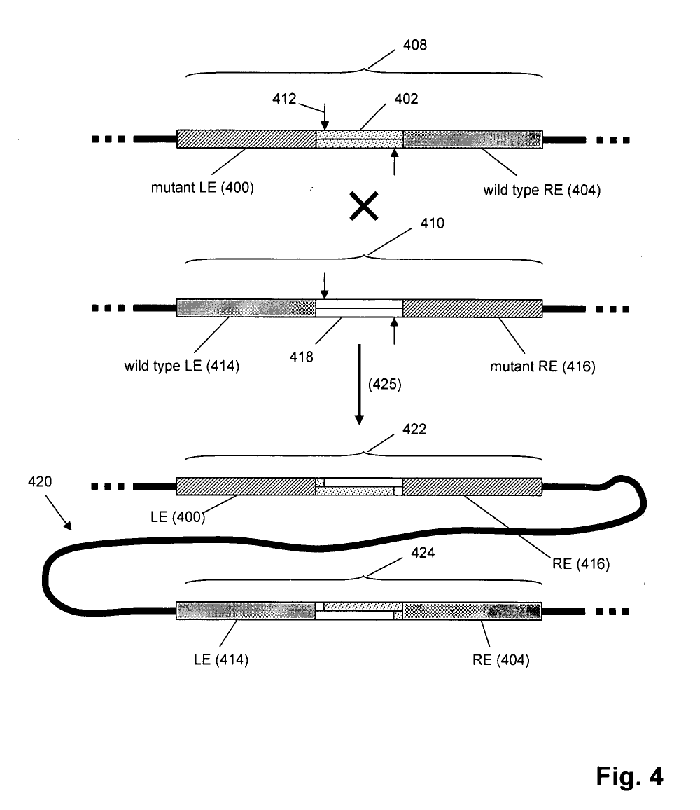

In one aspect, pairs of loxP sites may be used for assembling replacement genomes or large DNA circles in accordance with the invention. As illustrated in FIG. 4, loxP sites comprise a left end (400), i.e. “LE” Cre recognition site, or “arm,” a right end (404), i.e. “RE,” Cre recognition site, or “arm,” and sandwich between the LE and RE arms, a spacer region (402). In most wild type and mutant loxP sites, the LE and RE arms (400 and 404) are each 13 basepair in length, and the spacer region (402) is 8 basepairs in length. Also, in the wild type and in most mutant loxP sites, the LE and RE arms are inverted repeats. The components of the loxP site may be modified to produce sets of mutant loxP pairs, as illustrated in FIG. 4, which have the following properties: (i) members of a pair react with each other (i.e. to form recombinants), but essentially do not react with other member pairs of the set, and (ii) the product of a reaction between members of a pair are one inoperable loxP site (i.e., Cre is substantially unable to catalyze a recombination involving the site) one active loxP site (i.e., Cre is able to catalyze a recombination involving the site). In one aspect, the latter active loxP site is the wild type loxP site. Such pairs of loxP sites operate as illustrated in FIG. 4. There single mutant loxP site (408) recombines with single mutant loxP site (410) to produce recombinant (420) that has double mutant loxP site (422) and mutant-free loxP site (424). Single mutant loxP site (408) comprises mutant LE (400), wild type RE (402), and spacer region (402). Single mutant loxP site (410) comprises wild type LE (414), mutant RE (416), and spacer region (418). Spacer regions (402) and (418) usually (but not necessarily) have the same sequence within a pair of interacting (or compatible) sites. In one aspect, non-interacting loxP sites have spacer regions with different sequences. A Cre catalyzed recombination of mutant loxP sites (408) and (410) produces (425) a product (420) containing two separate loxP sites in which both mutant arms are brought together and both wild type arms are brought together. Mutant loxP sites are selected so that whenever a double mutant loxP is produced it is substantially inoperable with respect to further Cre catalyzed recombinations. This prevents undesired recombinations involving the sites when Cre is used in later steps of serial site-specific recombination. In another aspect, the second loxP site of recombinant (420) (which is usually the wild type loxP site) is fully active with other compatible loxP sites (e.g. that have the same spacer region). Thus, such sites may be used to add further segments to a replacement genome or pairs of such sites may be used to exchange fragments of a replacement genome, e.g. in a recombinase mediated cassette exchange (RMCE) type of reaction, Seibler and Bode, Biochemistry, 36: 1740-1747(1997); and Bode et al, U.S. Pat. No. 6,992,235; which references are incorporated by reference.

Many mutant loxP sites are available for use with the invention. For example, six mutant spacer sites that may be used with the invention have been described in the literature. e.g. Nucleic Acids Res. 14, 2287-2300 (1986)); Gene 216, 55-65 (1998); Nucleic Acids Res. 30, 3067-3077 (2002), and U.S. Pat. No. 6,465254, which are incorporated by reference. Additional loxP mutants may be obtained by various screening methods, e.g. as disclosed in Missirlis et al. (cited above); Langer et al, Nucleic Acids Research, 30: 3067-3077 (2002), and the like, which are incorporated by reference. Table I lists published loxP sites. The following formula provides a general description of pairs of loxP sites that may be used in the invention, e.g. as illustrated in FIGS. 1A-1E, wherein a first member of a pair is defined as:

LE₁-S₁-RE₁

and a second member of the pair is defined as:

LE₂-S₂-RE₂

where:

LE₁ is a mutant or wild type left end loxP site Cre recognition sequence and RE₁ is a mutant or wild type right end loxP site Cre recognition sequence such that whenever LE₁ is a wild type sequence, RE₁ is a mutant sequence, and whenever LE₁ is a mutant sequence, RE₁ is a wild type sequence;

LE₂ is a mutant or wild type left end loxP site Cre recognition sequence and RE₂ is a mutant or wild type right end loxP site Cre recognition sequence such that whenever LE₂ is a wild type sequence, RE₂ is a mutant sequence, and whenever LE₂ is a mutant sequence, RE₂ is a wild type sequence; with the proviso that whenever LE1 is a mutant sequence, then LE2 is a wild type sequence; and

S₁ and S₂ are compatible non-promiscuous loxP spacer regions. As used herein, “non-promiscuous” in reference to a loxP spacer sequence means that loxP sites containing such sequence (or pair of non-self recombining sequences) are substantially unreactive, or non-cross-reactive, with loxP sites containing other spacer sequences. In one aspect, non-promiscuous means that such sequence or pairs of sequences cross-react with less than 100 other loxP sites having a spacer selected from the set defined by formula NNNTANNN; in another aspect, such cross-reactivity is with less than 50 of such sites; in another aspect, such cross-reactivity is with less than of 20 such sites; and in another aspect, such cross-reactivity is with less than of 10 such sites.

In one aspect, LE, is the lox71 left end loxP site Cre recognition sequence whenever it is a mutant sequence and RE, is the lox66 right end loxP site Cre recognition sequence whenever it is a mutant sequence. Likewise, LE₂ is the lox71 left end loxP site Cre recognition sequence whenever it is a mutant sequence and RE₂ is the lox66 right end loxP site Cre recognition sequence whenever it is a mutant sequence.

In another aspect, S₁ and S₂ are both the same sequence selected from the group consisting of:

GTATAGTA GCGTATGT GGTTACGG
GGCTATAG TTGTATGG TTTTAGGT
TCGTAGGC GGATAGTA GAGTACGC
GTGTATTT AGGTATGC

In still another aspect, S₁ and S₂ are both the same sequence selected from the group consisting of:

GTATAGTA GCGTATGT GGTTACGG
GGCTATAG TTGTATGG TTTTAGGT
TCGTAGGC GGATAGTA GAGTACGC
GTGTATTT AGGTATGC

In another aspect, S₁ is GTGTACGC whenever S₂ is GTGTACGG; and S₂ is GTGTACGC whenever S₁ is GTGTACGG.

TABLE 1
(deviations from wild type shown in lower case)

| | | | | |
|---|---|---|---|---|
| | | | | SEQ ID |
| Site Name | LE | spacer | RE | NO |

| | | | | |
|---|---|---|---|---|
| wild type | ATAACTTCGTATA | ATGTATGC | TATACGAAGTTAT | 1 |
| lox511 | ATAACTTCGTATA | ATGTATaC | TATACGAAGTTAT | 2 |
| lox5171 | ATAACTTCGTATA | ATGTgTaC | TATACGAAGTTAT | 3 |
| lox2272 | ATAACTTCGTATA | AaGTATcC | TATACGAAGTTAT | 4 |
| m2 | ATAACTTCGTATA | AgaaAcca | TATACGAAGTTAT | 5 |
| m3 | ATAACTTCGTATA | taaTAcca | TATACGAAGTTAT | 6 |
| m7 | ATAACTTCGTATA | AgaTAgaa | TATACGAAGTTAT | 7 |
| mu | ATAACTTCGTATA | cgaTAcca | TATACGAAGTTAT | 8 |
| lox71 | taccgTTCGTATA | ATGTATGC | TATACGAAGTTAT | 9 |
| lox66 | ATAACTTCGTATA | ATGTATGC | TATACGAAcggta | 10 |

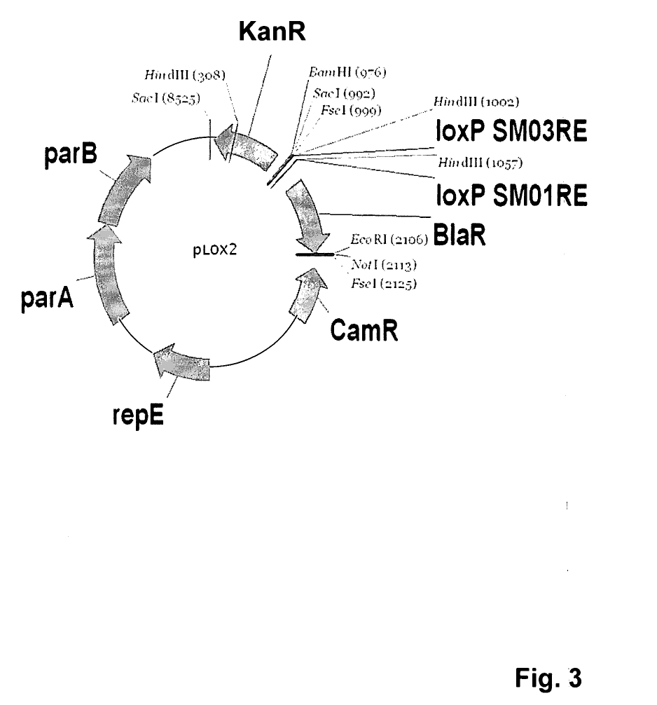

FIG. 3 is a genetic map of a representative pLOX vector that may be used for maintaining a plurality of segments for assembly into a replacement genome. Each vector has mutant loxP site for integration with a precursor replacement genome, and a recipient lox P site for receiving the next incoming clone in a subsequent assembly step. The replicon region of the vector is removed by FseI digestion prior to transformation.

### Kits of the Invention

In one aspect, kits of the invention comprise a plurality of vectors for accepting segments as inserts, each vector comprising at least one recombination element. Vectors for use with methods of the invention may each further include one or more selectable markers for determining the presence of a recombinant molecule. Kits of the invention may further include one or more recombinases to catalyze recombination reactions involving recombination elements in the vectors of the kits, as well as ancillary proteins, co-factors, and necessary buffers and salts for conducting recombination reactions. In one embodiment, kits of the invention include at least one Cre recombinase. Kits of the invention may further include reagents for selecting host organisms carrying desired recombinant molecules, including reagents for positive and/or negative selection.

Kits of the invention also include any delivery system for delivering materials or reagents for carrying out a method of the invention. In the context of recombination reactions for assembling a nucleic acid construct, such delivery systems include systems that allow for the storage, transport, or delivery of reaction reagents (e.g., vectors, enzymes, etc. in the appropriate containers) and/or supporting materials (e.g., buffers, written instructions for performing the reactions etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in a reaction, while a second container contains vectors.

### EXAMPLE I

### Use of H. influenzae as Genome Donor To An E. coli Host

In this example, H. influenzae was selected as a donor organism because of its free living status and its relatively compact genome (1.83 Mbp). The strain of H. influenzae employed is Rd KW20, which is kanamycin resistant and RecA negative (to eliminate the possibility of confounding homologous recombination). E. coli was selected as the host organism, specifically the HMS174 strain (Novagen), as it has K12 background, supports IPTG inducible recombinant protein expression, and is RecA negative. H. influenzae and E. coli are closely related, commensal gammaproteobacteria and the complete genome sequence is available for both organisms [(Science 269, 496-512 (1995); Science 277, 1453-1452 (1997)].

A BAC library was constructed from an MboI partial digest of H. influenzae gDNA. BAC clones from this library were end sequenced at high redundancy (>200× clone coverage) and mapped to the reference H. influenzea genome sequence. A minimal tiling path of 19 BAC clones that represents 95% of the H. influenzae genome and 98.6% of H. influenzae genes in intact predicted operons were selected. The tiling set was selected to maximize genome coverage, to minimize clone overlap, and to disrupt the fewest number of genes and predicted operons. This set of donor clones (i.e. segments) are used for re-assembly in the host organism. A 10 kb plasmid library was also constructed and end sequenced leading to the selection of 15 plasmids that can close most gaps in the BAC tiling path, if necessary.

Assays were carried out to assess the following: (i) H. influenzea genes that cannot be cloned in E. coli. There are at least 24 annotated genes present in the physical gaps that remain between clones in the minimal tiling set. (Generally, different minimal tiling sets may have different numbers of such genes). While there is no obvious pattern in this gene set of the presently selected minimal tiling set, there are several genes involved in galactose metabolism and also several uncharacterized genes, which may be dispensable. There are, however, five genes that encode what seem to be essential proteins, including three ribosomal proteins, a GTP-binding protein, and a DNA polymerase III subunit. These genes appear in the predicted set of 206 essential genes from a recent meta-analysis of all experimental and bioinformatic approaches undertaken to date that have attempted to define the core set of essential genes in a free living organism (Gil et al, cited above). While the non-essential toxic genes can likely be ignored, those that are toxic but essential are included in the replacement genome. Such genes may be of utility in resolving the donor and host genomes in the final hybrid organism. (ii) Which cloned H. influenzae genes are expressed in E. coli. A primary cDNA library was constructed from one of the BAC clones (M01) in the minimal tiling set which contained 135 predicted genes. A total of 9,216 ESTs were sequenced and a total of 123 unambiguous hits to Haemophilus genes were present in BAC M01. These ESTs collapsed into 17 clusters (i.e. 17 genes were represented). Thus, despite the limitations of this sampling procedure where most hits were against the host cell (E. coli) transcripts, it is clear that genes encoded in Haemophilus BACs are transcribed by the host cell machinery. A functional expression test was performed next. Surprisingly, the Haemophilus restriction systems appeared to be clonable in E. coli. This may occur due to the fact that restriction endonuclease genes are co-transcribed with a methylase gene that protects the cell from self-digestion. The HindIII restriction enzyme system is encoded by clone C09 in the minimal tiling set, and it was verified that genomic DNA extracted from this E.coli clone is in fact protected by expression of the HindIII methylase such that it is resistant to digestion by the HindIII restriction endonuclease.

### EXAMPLE II

### In vivo Assembly of Episomal Elements in E. coli

In this example episomal elements that contain mutant LoxP sites are constructed using standard molecular biology procedures. These constructs are transformed sequentially into an E. coli host and fusion is mediated by induction of Cre expression within the host cell. Separately, Bacterial Artificial Chromosomes (BACs) containing large segments of the H. influenzae genome are retrofit with mutant lox sites and selectable markers using the RED/ET system. BACs retrofitted in this manner are suitable for serial recombination in Cre expressing RED/ET E. coli host cells.

A short DNA segment with an EcoR1 compatible overhang on one end plus a HindIII compatible overhang on the opposite end, and containing a LoxP site that has both an LE arm mutant (ATAAC to TACCG) and a spacer mutant (C to G at spacer position 2 and A to C at spacer position 7) was ligated into EcoR1/HindIII cut and gel purified pET19b expression vector. This ampicillin resistant pET19b construct contains the coding sequence for the Cre enzyme inserted into the Nde1 site of the multiple cloning region, under control of an IPTG inducible promoter. This construct was transformed into HMS_{—}174 E. coli cells (Novagen), and a batch of electro-competent cells containing the pET19b construct was prepared. Separately, a complementary mutant LoxP site was designed that contained the same mutant spacer region as the above site, and a right element arm mutation (GTTAT to CGGTA). This site was inserted into the tetA (tetracycline resistance) gene in plasmid pBR322 by fusion PCR, using end primers tailed with Sac1 and Not1 restriction sites. The PCR product, comprised of the tetA gene with the embedded RE mutant loxP site was digested with Sac1 and Not and gel purified, then ligated into Sac1/Not1 digested and gel purified BAC vector pECBAC1. This construct was propagated in E. coli DH10B cells and DNA was isolated by alkaline lysis and transformed into the electro-competent HMS_{—}174 E. coli host cells already harboring the pET19b LE mutant LoxP construct. Cells were grown first on solid media containing IPTG and ampicillin, then harvested and transferred to plates containing tetracycline and fusaric acid. Fusaric acid is toxic to cells expressing a functional tetA gene. Since the pECBAC1 construct contains a RE mutant LoxP site embedded in the tetA gene, upon Cre mediated recombination that joins the pECBAC1 and pET19b constructs, the pECBAC1 tetA insert is disrupted, allowing recombinants to grow on fusaric acid containing solid media. A total of 96 clones were screened for recombination by colony PCR using a left primer complementary to the Cre gene in the pET19b construct and a right primer complementary to a region of the tetA gene in the pECBAC1 construct. The expected junction fragment was observed in two of the 96 clones screened. Subsequent sequencing of these positive clones indicated a fusion of the tetA and Cre genes and conversion of the single arm mutant loxP sites to a wild type site, thus verifying successful in vivo recombination.

The RED/ET system was used to retrofit a BAC (BAC I) containing a large (104 kbp) segment of the H. influenzae genome with a mutant LoxP site. A double stranded oligonucleotide was prepared that comprised a LoxP site with the LE arm mutation (ATAAC to TACCG), a wild type spacer region, and EcoR1 compatible overhang on one end plus a HindIII compatible overhang on the opposite end. This oligonucleotide was ligated into EcoR1/HindIII cut and gel purified pET19b vector. Subsequently, a segment of this pET19b construct containing the beta-lactamase (bla) gene for ampicillin resistance next to the inserted LE mutant LoxP site was amplified by PCR. The PCR primers were tailed with 44 bp sequences homologous to a non-essential segment of the backbone of the pECBAC1 vector. The H. influenzae BACI was transformed into electro-competent EL350 cells. These cells carry the Cre gene under control of an arabinose inducible promoter, and a segment of the bacteriophage lambda genome encoding the exo, bet and gam genes under control of a temperature sensitive repressor (Yu et al. PNAS, 2000). Heat induced, electro-competent EL350 cells carrying the 104 kbp H. influenzae segment in chloramphenicol-resistant pECBAC1 were prepared and transformed with the PCR product containing the bla gene, mutant lox site and pECBAC1 homology arms. Transformed cells were plated on solid media containing chloramphenicol and ampicillin. Numerous colonies were picked and targeted insertion of the mutant LoxP cassette was sequence verified.

For H. influenzae precursor genome assembly, the neighboring clone in the H. influenzae BAC minimal tiling path (BAC II) is retrofit with a cassette containing a complementary LoxP site. A double stranded oligonucleotide is prepared that comprises a LoxP site with the RE arm mutant (GTTAT to CGGTA), a wild type spacer region, and Spe1 compatible overhangs. This oligonucleotide is ligated into Spe1 cut and gel purified pGPS1.1 vector. Subsequently, a segment of this pGPS1.1 construct containing the kanamycin resistance gene next to the inserted RE mutant LoxP site is amplified by PCR. The PCR primers are tailed with the 44 bp sequences, as above, that are homologous to a non-essential segment of the backbone of the pECBAC1 vector. BACII is transformed into electro-competent EL350 cells and heat induced, electro-competent EL350 cells carrying the H. influenzae genome segment in chloramphenicol-resistant pECBAC1 are prepared, followed by transformation of the PCR product containing the kanamycing resistance gene, RE mutant lox site and pECBAC homology arms and integration of this cassette into BACII.

Cells containing RE loxP retrofit BACII are grown and BAC DNA is isolated by standard alkaline lysis procedures and purified by pulsed field gel electrophoresis (PFGE). Electro-competent EL350 cells containing LE loxP retrofitted BACI are prepared, and transformed with purified retrofitted-BACII DNA and grown in liquid media in the presence of L(+)-arabinose for induction of Cre gene expression plus double antibiotics (ampicillin and kanamycin) which are necessary to maintain both retrofit-BACI and retrofit-BACII in the host cells to allow their recombination. The F replicon in the BAC vector maintains stringent copy number control such that only one or two copies are present per cell. Cells are plated and colonies picked and screened for the presence of the BAC1/BACII fusion, which represents the first recombinant precursor genome in the modular genome construction process. The orientation of the two genome segments in the consolidated BAC is predetermined by defining the orientation of the spacer regions of the LoxP sites introduced into the original BACs, BACI and BACII. Post recombination, the consolidated circular DNA molecule (BACI/II) will have one reactive wild type LoxP site, an inert double arm mutant LoxP site, an amplicillin resistance gene, a kanamycin resistance gene and two juxtaposed copies of the pECBAC1 vector backbone. Using RED/ET recombination, the wild type LoxP site and the adjacent antibiotic resistance marker are replaced with a new cassette that contains a new LE arm mutant loxP site and a previously unused antibiotic resistance marker, such as the zeocin resistance gene or the gentamycin resistance gene. The next adjacent clone in the tiling path (BACIII) is retrofit as above with a complementary RE arm mutant site and an antibiotic resistance gene, which may be the antibiotic resistance gene removed from BACI/II or a different antibiotic resistance gene. DNA is prepared from BACIII and transformed into host cells harboring BACI/II. Cells are grown in liquid media in the presence of L(+)-arabinose for induction of Cre gene expression and the appropriate double antibiotics to maintain both BACI/II and BACIII in the host cells, so as to allow their recombination. In this manner, each BAC in the tiling path is added to the growing precursor genome until the genome is tiling path is completely reassembled, or until a desired state of re-assembly is achieved.

### EXAMPLE III

### In vivo Assembly of Large DNA Circles in E. coli Using Fosmid Large-Insert Vectors and a Lambda Red Recombination System

This example discloses a vector system for efficient assembly of large DNA molecules by iterative in vivo recombination of fosmid clones. Using this technique two non-contiguous regions of the Haemophilus influenzae genome has been assembled as an episome in recombinogenic Escherichia coli host cells. These regions together comprise 190 kbp, or 10.4% of the H.influenze genome. The lambda Red system (Yu et al., cited above) is an efficient and scarless method of in vivo recombination. This system utilizes a host strain (typically E.coli DH10B cells) carrying a segment of the phage lambda genome that contains the exo, bet and gain genes under control of a temperature-sensitive repressor. These lambda genes mediate recombination between the ends of a linear incoming DNA segment with homologous sequences in a target DNA. The homology regions can be very short (˜50 bp) and the target can be any chromosomal or episomal DNA molecule present in the host cell. A fosmid vector system is disclosed below that allows lambda Red recombinations to be done iteratively, such that large DNAs can be assembled stepwise in the host cell. Such iterative recombination was demonstrated by reassembling segments of the H.influenzae Rd KW20 genome in an E.coli host.

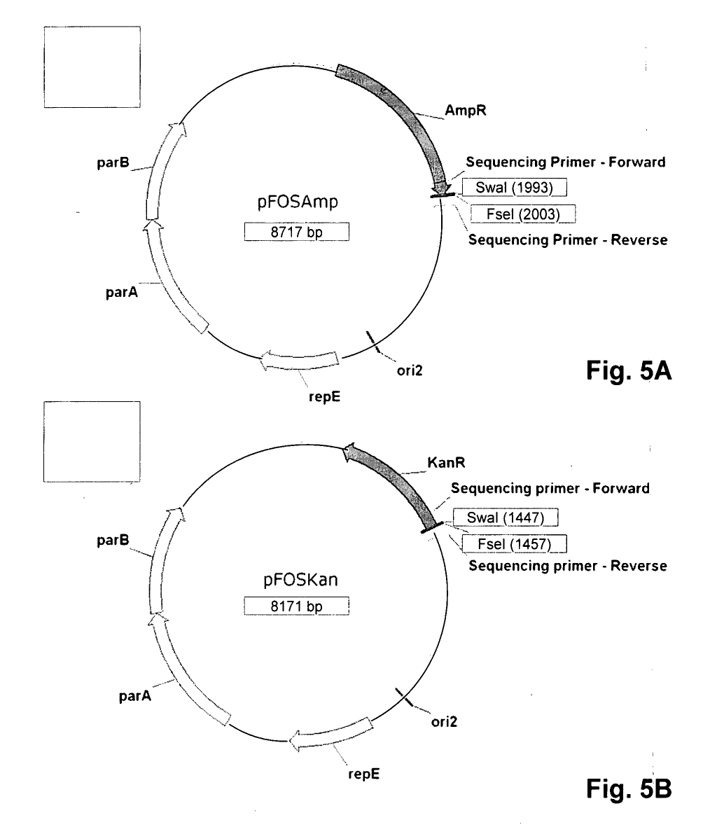

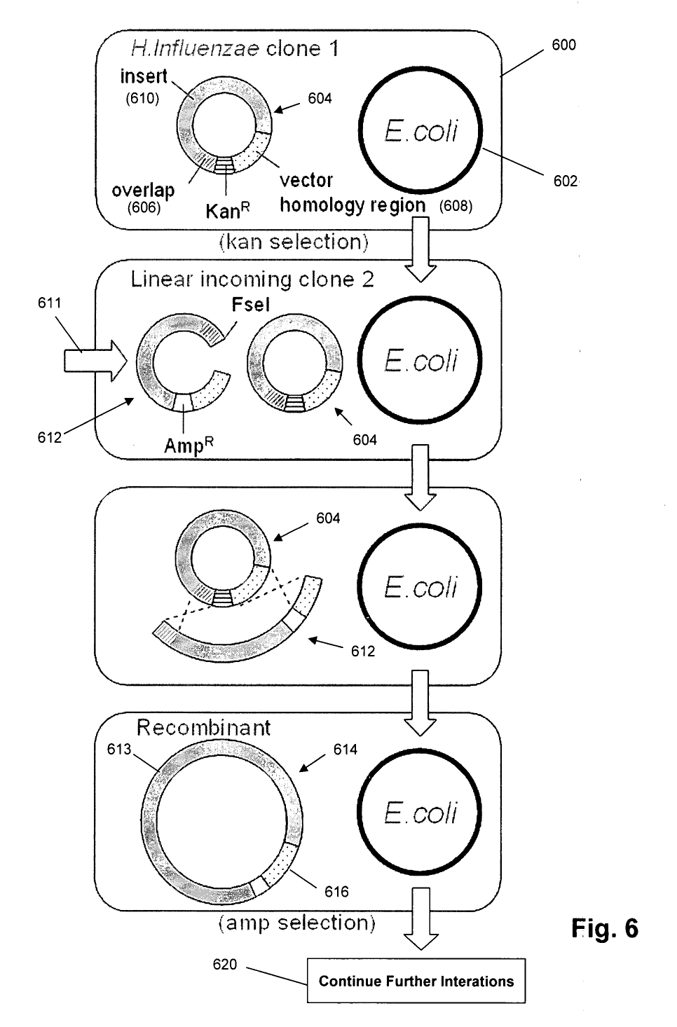

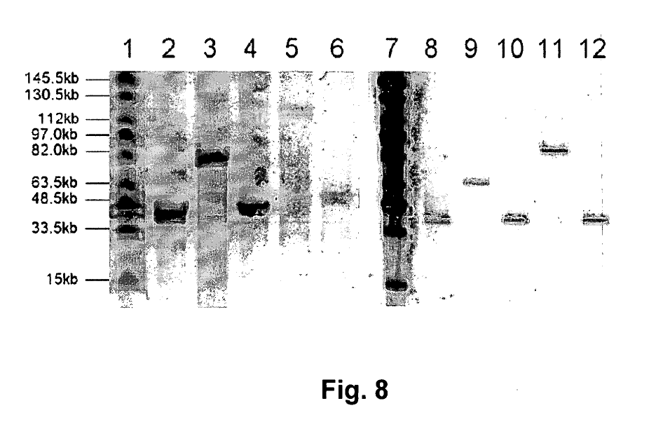

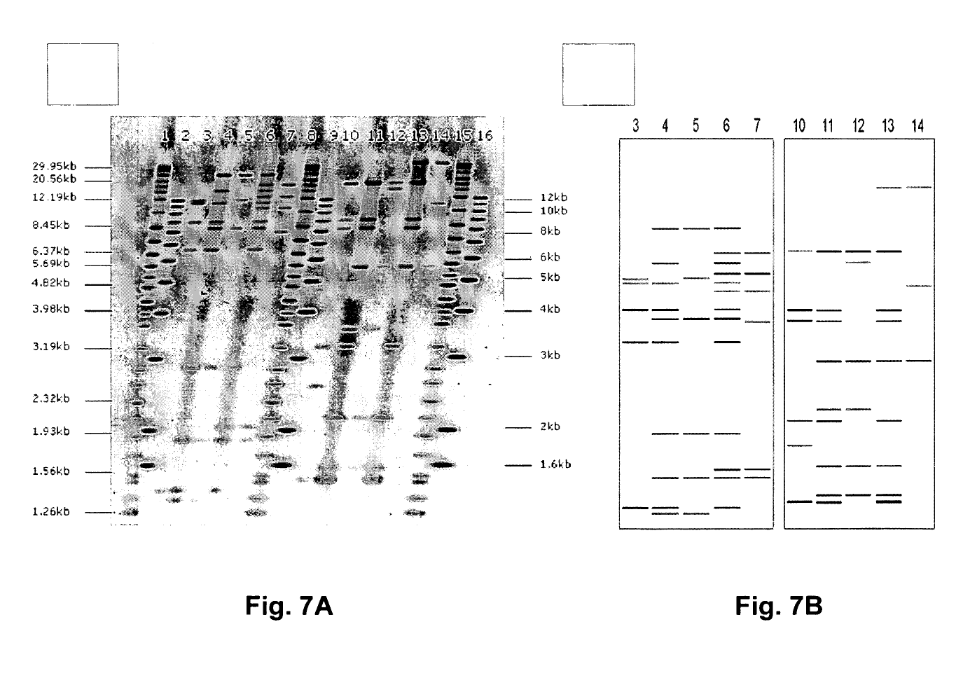

Two fosmid vectors, one containing an ampicillin resistance marker (pFOSAMP, FIG. 5A) and the second a kanamycin resistance marker (pFOSKAN, FIG. 5B), were constructed as described below by modifying the pEPIFOS5 fosmid vector backbone (EpiCentre Biotechnologies, Madison, Wis.). A unique FseI restriction site was engineered to the right of the insert site and serves to linearize the clones that are to recombine with the target construct (e.g. precursor genome) in the host. Two H.influenzae fosmid libraries were built, one in each vector, and random clones were end-sequenced to high redundancy. End-sequences were mapped to the H.influenzae Rd KW20 reference genome sequence and repeats within and between H.influenzae and E.coli were highlighted to facilitate selection of a minimal tiling set (i.e. an ordered plurality) of clones with non-repetitive end sequences. The selected minimal tiling set included 61 clones, 31 from the pFOSAMP library and 30 from the pFOSKAN library and covers 98.5% of the 1.83 Mbp H.influenzae genome. Two non-contiguous genomic regions were reassembled using the method illustrated in FIG. 6. Three overlapping fosmids were selected for each region, and the first fosmid (604) having insert (610) was transformed into lambda Red compatible E.coli cells (600) having genome (602), cultured in the presence appropriate antibiotic (eg. ampicillin), and electrocompetent cells were prepared. The next clone in the tiling path (612) was linearized by FseI, end polished, then transformed (611) into the cells containing the initial fosmid. The incoming clone was joined with the first clone by a recombination event mediated at one end by the vector sequence (608) and at the other end by overlapping H.influenzae genomic sequence (606) to produce recombinant (614). A key feature of this system is that upon recombination, the new genomic segment is joined to the first to produce insert (613), but at the same time the vector segment from the first clone is replaced with the vector segment of the second clone. As such, the product contains only a single vector sequence (616), but now the antibiotic resistance marker has been exchanged and recombinant clones can be selected. Importantly, the incoming clone cannot propagate on its own because it is linear, and only becomes circularized (and thus replicable) upon recombination with the first resident clone. A second round of recombination was then undertaken with the third fosmid clone for each region, to generate large contiguous H.influenzae genomic DNA segments propagating in their E.coli hosts. The size and content of the intermediate and final constructs was verified by EcoRI restriction mapping (FIG. 7A) and also by sizing the FseI cut gDNA by pulsed field gel electrophoresis (FIG. 8, described below). End sequences of each final construct verify the reassembly of test segment 1 (bases 446, 461-553, 659) and test segment 2 (bases 63,192 to 145,915 bp) of the H.influenzae chromosome.

### A. Vector Construction.

Two fosmid vectors with different antibiotic resistance genes were constructed. pEpiFOS5 (Epicentre), a derivative of BAC vector pBeloBAC11 was digested with Eco721 and ScaI to remove a 500 bp segment between these sites that showed exact homology to the E.coli genome (and could, therefore, interfere with recombination). The 500 bp segment was replaced by a DNA segment comprised of an FseI restriction site, a SwaI restriction site, and either the ampicillin resistance gene (from plasmid pET19b) or the kanamycin resistance gene (from TN7). SwaI is the insert site, and the FseI site is used to linearize the vector prior to recombination. The correct assembly of these two new vectors, pFOSAMP and pFOSKAN was verified by sequencing.

### B. Library Construction.

H.influenzae Rd KW20 cells were cultured overnight in brain-heart infusion broth supplemented with hemin (10 ug/mL) and nicotiniamide adenine dinucleotide (100 ug/mL). Cells were pelleted and resuspended in Lysis Solution (10 mM Tris-Cl pH8, 100 mM EDTA pH8, 0.5% (w/v) SDS, 20 ug/mL RNase A, Proteinase K 100 ug/mL and incubated in a 50C bath, 3 h, mixing by gentle inversion every hour. Lysate was extracted three times with equal volumes of phenol:chloroform:isamyl alcohol (25:24:1) then ethanol precipitated, spooled, and dissolved in TE. DNA was hydrodynamically sheared with a 25 guage needle and a 25-40 kbp size fraction was isolated by pulsed field gel electrophoresis. Using the EpiFos Fosmid Library Production Kit (Epicentre), size-selected H.influenzae DNA was cloned into Swa1-linearized and dephosphorylated pFOSAMP and pFOSKAN vectors, then packaged and plated on 2XYT agar containing the appropriate antibiotic, according to the manufacturer's instructions.

### C. Clone Mapping.

Fosmid end sequences were obtained using custom primers (pFOSKAN_forward 5′>GAGCATTACGCTGACTTGAC (SEQ ID NO: 11); pFOSAMP_forward 5′>ACGATAGTTACCGGATAAGG (SEQ ID NO: 12); reverse 5′>CAAATATTATACGCAAGGCG (SEQ ID NO: 13)) and previously described nanolitre scale Sanger sequencing methods, e.g. Smailus et al, Genome Research, 15: 1447 (2005). A total of 11,520 total fosmid paired end sequences (5760 from each library) were obtained and these were vector-trimmed using cross_match [Green P (1994) www.phrap.org] and quality trimmed using trim2 (-M 10) (Huang et al, Genome Research, 13: 2164 (2003)). The resulting 9,935 sequences (5034 from the pFOSAMP library and 4901 from the pFOSKAN library) were aligned to the completed H.influenzae genome strain Rd KW20 using wuBLASTn [blast version 2.0, May 10, 2005; http://blast.wustl.edu]. The default parameters were used and only the best scoring match from each fosmid read with alignments longer than 200 nucleotides and sharing more than 70% sequence identity with the reference genome were subsequently evaluated. Pairing logic was respected while mapping fosmid end-reads, with pairs from any given clones aligning in opposite directions, facing inwards. Pairs aligning outside 40 kbp+/−2 SD of the insert size distribution were not considered. Custom software was designed to aid in mapping the genomic constructs onto the complete H.influenzae genome sequence and to help identify suitable candidates for the minimal tiling set.

### D. Selecting a Minimal Tiling Set.

Because our iterative recombination scheme is directional, all tiling path clones must have inserts that map to the same strand (eg. they must be in the same orientation with respect to vector). Higher coverage (99.21%) was obtained for the plus strand than the minus strand, so clones for the minimal tiling set were selected from the plus strand. The following rules were established for selecting a minimum set of clones; 1) overlapping clones must have alternate selectable markers, 2) the 3′-most 50-100 bp of the linearized incoming clone must not align to the E.coli genome, or to any repeats within the H.influenzae genome, and 3) clone inserts should provide maximal genome coverage and show minimal overlap, with a suitable overlap ˜500-10,000 bp, but no smaller than 50 bp. Intra and inter chromosomal repeats were detected using cross match (Green, P. 1994. www.phrap.org) and only repeats larger than 20 bp and having more than 70 sequence identity were considered. Selection was performed in a semi-automated fashion, whereby suitable clones were flagged by software written in-house and validated manually, putting emphasis on the uniqueness of 3′ end sequences while ensuring a maximal clone overlap for mediating recombination. The final minimal tiling set included 61 clones, 31 from the pFOSAMP library and 30 from the pFOSKAN library. The tiling set includes three gaps (1.7 kb, 11.4 kb and 15.3 kb) and covers 98.5% of the 1.83 Mbp H. influenzae genome.

### E. Iterative Clone Assembly.

Two regions of the genome were reconstructed. For each region, the first of the three fosmids to be assembled was transformed into EL350 cells, which harbor the prophage encoding lambda recombination proteins exo, bet and gain under control of the c1857 temperature sensitive transcriptional repressor. These cells were cultured, heat-shocked for 15 minutes at 42° C., then immediately cooled on ice and made electrocompetent by washing three times with ice-cold 10% glycerol. The second clone in each set of three was linearized by FseI digestion, end trimmed (to remove the residual bases of the FseI site) by Bal31 exonuclease digestion and then polished with T4 polymerase and T4 polynucleotide kinase to give phosphorylated blunt ends. Approximately 1 μg of linearized end-polished fosmid DNA was transformed by electroporation and recombinants were selected by plating on 2XYT media containing antibiotic matching the resistance marker on the incoming linearized clone. This procedure was repeated for the third clone in each set.

FIG. 7A shows EcoR1 restriction maps of individual fosmid clones that are combined to form larger contiguous DNA inserts. There are three fosmid clones covering each of two regions of the H.influenzae genome. For each set of three fosmids, two iterative recombination events are required for assembly. FIG. 7A shows EcoR1 digests of the initial fosmids (lanes 3, 5 and 7 for region 1, and lanes 10, 12 and 14 for region 2) used in reconstruction. Digests of intermediate clones created by fusing the first two clones in each set are shown in lane 4 (fusion of clones from lanes 3 and 5) and lane 11 (fusion of clones from lanes 10 and 12). Digests of the final constructs created by fusing each intermediate clone with the third and final clone in each set are shown in lane 6 (fusion of clones from lanes 5 and 7) and lane 13 (fusion of clones from lanes 12 and 14). Lanes 1, 2, 8, 9, 15 and 16 contain size markers. FIG. 7B shows the expected banding patterns from in-silico digestion of the constructs from FIG. 7A. The lanes in FIG. 7B match those in FIG. 7A. Grey bands in FIG. 7B are restriction fragments that contain vector DNA.

FIG. 8 shows pulsed field gel electrophoresis sizing of FseI linearized precursor and assembled clones. There are three initial fosmid clones that are assembled into two large constructs covering test segment one (446, 461-553, 659 bp) and test segment two (63, 192-145, 915 bp) of the H.influenzae genome, respectively. For each set of three precursor fosmids, two iterative recombination events are required for assembly. For test segment one, FseI-linearized DNA for precursor fosmids is in lanes 2, 4 and 6. The intermediate construct for segment 1, created by fusing the first two clones (lanes 2 and 4), is shown in lane 3, and FseI-linearized DNA for tile final construct, created by fusing the intermediate (lane 3) construct with the third and final clone (lane 6) is shown in lane 5. For test segment two, FseI-linearized DNA for precursor fosmids is in lanes 8, 10 and 12. The intermediate construct for segment 2 created by fusing the first two clones for this segment (lanes 8 and 10) is shown in lane 9, and FseI-linearized DNA for the final construct for test segment 2, created by fusing the intermediate construct (lane 9) with the third and final clone (lane 12) is shown in lane 11. High molecular weight DNA markers are in lanes 1 and 7.

### DEFINITIONS

Terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g. conditions including temperature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1 989) and Anderson “Nucleic Acid Hybridization” 1^{st} Ed., BIOS Scientific Publishers Limited (1999), which are hereby incorporated by reference in its entirety for all purposes above. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Inducible” or “inducible control” in reference to gene expression means that gene expression is controlled by a promoter and possibly of regulatory elements such that a promoter is transcription ally active under a specific set of conditions, e.g., a change in physical conditions, such as a change in pH, temperature, salt concentration, or the like, or the presence of a particular chemical signal or combination of chemical signals that, for example, affect binding of the transcriptional activator to the promoter and/or affect function of the transcriptional activator itself.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Nucleic acid construct” is used synonymously with “recombinant DNA molecule.”

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6:855-870 (1996); Mesmaeker et al, Current Opinion in Structural Biology, 5:343-355(1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. Reaction volumes typically range from a few hundred nanoliters, e.g. 200 nL, to a few hundred μL, e.g. 200 μL. “Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Recombination element” means a sequence that is a site of recombination of DNA sequences in a recombination reaction. A recombination element may be a segment of DNA that is homologous to another segment that participates in a recombination reaction (e.g. as in homologous recombination), or it may be a specific sequence where recombination takes place by action of an associated recombinase, and perhaps additional ancillary factors, that recognizes all or part of the specific sequence (e.g. as in site-specific recombination). In one aspect, a recombination element is a recombination site of a site-specific recombination system, such as Cre-LoxP, Flp-FRT, or the like. In another aspect, a recombination element is a region of sequence homology between two pieces of DNA that undergo recombination in the presence of a homologous recombination system, such as a lambda Red recombination system, or like system. In reference to recombination elements that undergo recombination in accordance with the invention, “sequence homology” means an effective degree of sequence similarity between two recombination elements for detectable recombination to occur for a recombination system being employed, e.g. as determined by a selective marker under conventional screening conditions. In one aspect, recombination elements for use in the method of the invention have identical sequences.

“Regulatory elements” in reference to gene expression means DNA sequences that are operably linked to the expression of one or more genes. Such elements are commonly located at positions adjacent to the expressed genes and can include promoters, terminators, antiterminators, activators, attenuators, and the like, e.g. Kornberg and Baker, DNA Replication, 2^{nd} Edition (Freeman, San Francisco, 1992), Makrides, Microbiological Reviews, 60: 512-538 (1996). Frequently, one or more co-regulated genes are associated with the same set of regulatory elements in an operon.

“Synthetic” in reference to a polynucleotide segment of the invention means that all or a portion of the segment is constructed from one or more polynucleotides that were initially chemically synthesized. After synthesis, such synthetic polynucleotide segments may be replicated by in vivo or in vitro enzymatic methods, e.g. by conventional cloning or by amplification, such as by PCR, RCR, or the like. Various approaches may be used to constructing synthetic polynucleotide segments for use with the invention including, but not limited to, those described in the following references that are incorporated herein by reference: Tian et al, Nature, 432: 1050-1054 (2004); Soldatov et al, International patent publication WO 2004/092375; U.S. patent publication 2003/0138782A1; U.S. patent publication 2003/0165946A1; U.S. patent publication 2005/0106606A1; International patent publication WO 2006/044956; Cleary et al, Nature Methods, 1: 241-248 (2004); Zhou et al, Nucleic Acids Research, 32: 5409-5417 (2004); Chen et al, J. Chem. Soc., 116: 8799-8800 (1994); Mandecki et al, Gene, 68: 101-107 (1988); Kodumal et al, Proc. Natl. Acad. Sci., 101: 15573-15578 (2004); Smith et al, Proc. Natl. Acad. Sci., 100: 15440-15445 (2003); or the like.

What is claimed is:

1. A method of assembling a replacement genome in a host organism having a host genome, the method comprising the steps of:
(a) providing a plurality of segments that cover a replacement genome, each segment being associated with one or more recombination elements, wherein at least one of such recombination elements comprises a portion of the segment;
(b) transforming or co-transforming the host organism with one or more segments to form a precursor genome, the precursor genome having a region homologous to the portion of the at least one recombination element, and the precursor genome being a recombinant of the one or more segments or a recombinant of a prior precursor genome and the one or more segments, such recombinant being formed by recombination of the recombination elements associated with the one or more segments, wherein such recombination includes recombination of the portion of the at least one recombination element and the corresponding homologous region of the precursor genome;
(c) repeating step (b) with segments of a predetermined ordering until the replacement genome is formed; and
(d) removing the host genome.

2. The method of claim 1 wherein said step of repeating comprises repeating said step (b) with each of said predetermined ordering of said plurality of said segments.

3. The method of claim 2 wherein one of said segments includes an origin of replication operable in said host organism.

4. The method of claim 3 wherein each of said segments includes a selectable marker that permits identification of said host organisms that contain a recombinant of a segment and a prior precursor genome.

5. The method of claim 4 wherein said host organism is a prokaryotic organism and wherein said donor organism is a prokaryotic organism and wherein each of said segments is carried by a separate large-insert vector.

6. The method of claim 5 wherein said host organism is Escherichia coli and said recombination is implemented by a lambda Red recombination system.

7. The method of claim 1 wherein said step of removing includes transforming a separate host organism with said replacement genome.

8. A method of forming in a host organism a DNA circle comprising an ordered plurality of polynucleotides, the method comprising the steps of:
(a) providing an ordered plurality of polynucleotides, each polynucleotide being associated with one or more recombination elements, wherein at least one of such recombination elements comprises a portion of the polynucleotide;
(b) transforming or co-transforming the host organism with one or more polynucleotides to form a precursor DNA circle, the precursor DNA circle having a region homologous to the portion of the at least one recombination element, and the precursor DNA circle being a recombinant of the one or more polynucleotides or a recombinant of a prior precursor DNA circle and the one or more polynucleotides, such recombinant being formed by recombination of the recombination elements associated with the one or more polynucleotides, wherein such recombination includes recombination of the portion of the at least one recombination element and the corresponding homologous region of the precursor DNA circle;
(c) repeating step (b) with polynucleotides of the ordered plurality of polynucleotides until the DNA circle is formed.

9. The method of 8 wherein said step of repeating comprises repeating step (b) with each of said ordered plurality of said polynucleotides until said DNA circle is formed.

10. The method of claim 9 wherein each of said polynucleotides includes a selectable marker that permits identification of said host organisms that contain a recombinant of a polynucleotide and a prior precursor DNA circle.

11. The method of claim 10 wherein said step of transforming further includes selecting said host organisms that are identified as containing said recombinant by said selectable marker.

12. The method of claim 11 wherein each of said polynucleotides is carried by a separate large-insert vector each having a vector region and wherein another of said one or more recombination elements comprises a portion of the vector region.

13. The method of claim 12 wherein said host organism is a prokaryotic cell.

14. The method of claim 13 wherein two or more of said polynucleotides of said ordered plurality have a length greater than 300 kilobases.

15. The method of claim 12 wherein said host organism is Escherichia coli and wherein said recombination is implemented with a lambda Red recombination system.

16. The method of claim 12 wherein said host organism is a eukaryotic cell.

17. A host organism containing a DNA circle comprising an ordered plurality of polynucleotides, the DNA circle produced by the steps of:
(a) providing an ordered plurality of polynucleotides, each polynucleotide being associated with one or more recombination elements, wherein at least one of such recombination elements comprises a portion of the polynucleotide;
(b) transforming or co-transforming the host organism with one or more polynucleotides to form a precursor DNA circle, the precursor DNA circle having a region homologous to the portion of the at least one recombination element, and the precursor DNA circle being a recombinant of the one or more polynucleotides or a recombinant of a prior precursor DNA circle and the one or more polynucleotides, such recombinant being formed by recombination of the recombination elements associated with the one or more polynucleotides, wherein such recombination includes recombination of the portion of the at least one recombination element and the corresponding homologous region of the precursor DNA circle;
(c) repeating step (b) with polynucleotides of the ordered plurality of polynucleotides until the DNA circle is formed.

18. The host organism of claim 17 wherein said step of repeating comprises repeating said step (b) with each of said ordered plurality of polynucleotides until said DNA circle is formed.

19. The host organism of claim 18 wherein each of said polynucleotides includes a selectable marker that permits identification of said host organisms that contain a recombinant of a polynucleotide and a prior precursor DNA circle.

20. The host organism of 19 wherein said step of transforming further includes selecting said host organisms that are identified as containing said recombinant by said selectable marker.

21. The host organism of claim 20 wherein said host organism is a prokaryotic cell and wherein each of said polynucleotides is carried by a separate large-insert vector each having a vector region.

22. The host organism of claim 21 wherein said host organism is a bacteria.

23. The host organism of claim 21 wherein another of said one or more recombination elements comprises a portion of the vector region.

24. The host organism of claim 23 wherein two or more of said polynucleotides of said ordered plurality have a length greater than 300 kilobases.

25. The host organism of claim 23 wherein said host organism is Escherichia coli and wherein said recombination is implemented by a lambda Red recombination system.

26. The host organism of claim 19 wherein said host organism is a eukaryotic cell and wherein each of said polynucleotides is carried by a separate large-insert vector each having a vector region.

27. The host organism of claim 26 wherein said host organism is a yeast.

28. The host organism of claim 26 wherein two or more of said polynucleotides of said ordered plurality have a length greater than 300 kilobases.


SEQUENCE LISTING (13 sequences)

SEQ ID NO: 1 - DNA, 34 bp, Escherichia coli
ataacttcgtataatgtatgctatacgaagttat

SEQ ID NO: 2 - DNA, 34 bp, Escherichia coli
ataacttcgtataatgtatactatacgaagttat

SEQ ID NO: 3 - DNA, 34 bp, Escherichia coli
ataacttcgtataatgtgtactatacgaagttat

SEQ ID NO: 4 - DNA, 34 bp, Escherichia coli
ataacttcgtataaagtatcctatacgaagttat

SEQ ID NO: 5 - DNA, 34 bp, Escherichia coli
ataacttcgtataagaaaccatatacgaagttat

SEQ ID NO: 6 - DNA, 34 bp, Escherichia coli
ataacttcgtatataataccatatacgaagttat

SEQ ID NO: 7 - DNA, 34 bp, Escherichia coli
ataacttcgtataagatagaatatacgaagttat

SEQ ID NO: 8 - DNA, 34 bp, Escherichia coli
ataacttcgtatacgataccatatacgaagttat

SEQ ID NO: 9 - DNA, 34 bp, Escherichia coli
taccgttcgtataatgtatgctatacgaagttat

SEQ ID NO: 10 - DNA, 34 bp, Escherichia coli
ataacttcgtataatgtatgctatacgaacggta

SEQ ID NO: 11 - DNA, 20 bp, Escherichia coli
  primer (1)..(20)
gagcattacgctgacttgac

SEQ ID NO: 12 - DNA, 20 bp, Escherichia coli
  primer (1)..(20)
acgatagttaccggataagg

SEQ ID NO: 13 - DNA, 20 bp, Escherichia coli
  primer (1)..(20)
caaatattatacgcaaggcg